(19) United States
(12) Patent Application Publication
(10) Pub. No.: US 2009/0311710 A1
(43) Pub. Date: Dec. 17, 2009

(54) MUTAGENESIS METHODS USING RIBAVIRIN AND/OR RNA REPLICASES

(76) Inventors: Gregory COIA, Brunswick (AU); George Kopsidas, Preston (AU); Merilyn Sleigh, Neutral Bay (AU)

Correspondence Address: FROMMER LAWRENCE & HAUG, 745 FIFTH AVENUE- 10TH FL., NEW YORK, NY, 10151, US

(21) Appl. No.: 12/503,539

(22) Filed: Jul. 15, 2009

Related U.S. Application Data
(63) Continuation of application No. 11/115,001, filed on Apr. 26, 2005, now abandoned.
Application No. 11/115,001 is a continuation-in-part of application No. PCT/AU2003/001455, filed on Nov. 3, 2003.

(30) Foreign Application Priority Data
Nov. 1, 2002 (AU) 2002952432
Jun. 13, 2003 (AU) 2003902957
May 13, 2004 (WO) 2004/039995

(51) Int. Cl. C12Q 1/68

(52) U.S. Cl. 435/6

(57) ABSTRACT

The use of RNA-replicases for introducing mutations into and selecting for improved RNA molecules is described. The use of ribavirin, or a derivative/analogue thereof, in methods for introducing one or more mutations during replication or transcription of a target nucleic acid molecule is also described. These methods can be used to screen for nucleic acids, or proteins encoded thereby, with altered or new activity. Also provided are kits comprising ribavirin, or a derivative/analogue thereof, for use in mutagenesis procedures.

### CROSS REFERENCE TO RELATED APPLICATIONS

This application is a continuation-in-part of International Application No. PCT/AU2003/001455, filed Nov. 3, 2003, published as WO 2004/039995 on May 13, 2004, and claiming priority to Australian Application Nos. 2002952432, filed Nov. 1, 2002 and 2003902957, filed Jun. 13, 2003.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to methods of incorporating mutations into a nucleic acid molecule. In one aspect, the invention relates to the use of RNA-replicases for introducing mutations into RNA and selecting for improved RNA molecules. In another aspect, the present invention relates to the use of ribavirin, and related nucleoside and nucleotide analogues, as a means of introducing mutations into nucleic acid molecules. The methods can be used, inter alia, for in vitro evolution of RNA, DNA and proteins, and in processes for the production and selection of improved RNA molecules or protein variants with diagnostic or therapeutic utility.

### BACKGROUND OF THE INVENTION

### Evolution of RNA Molecules

RNA molecules carry out a number of important functions in biological systems. For example, RNA molecules act as:

(i) genomes for some classes of virus and bacteriophage;

(ii) messenger RNA molecules to carry the coding information for protein synthesis;

(iii) tRNA molecules, as amino acid carriers in protein synthesis;

(iv) structural molecules, as part of ribosome and nuclear complexes;

(v) regulatory molecules, such as naturally occurring ribozymes, and RNAs that play a role in RNA splicing; and

(vi) artificial regulators, such as introduced ribozymes, antisense RNAs and interfering RNAs.

The functionality of all RNA molecules is determined by a combination of primary structure (nucleotide sequence) and secondary and tertiary structure (folding and association). Nucleotide sequence is the major determinant of other RNA properties including not only folding but also stability, translatability and recognition by binding proteins and other molecules.

There have been a number of reports in the scientific literature of naturally occurring or artificially generated changes to RNA molecules that influence biological function, and these in turn have helped to identify the sequences and structures important for maintaining such functions.

For example, naturally occurring ribozymes from Tetrahymena fold into complex structures that are important for their stability and activity. It has been shown that mutations in the ribozyme sequence can influence the rate of folding by up to 50 fold (Deras and Woodson, 2000). Such mutations stabilise the folded molecules, increasing thermal stability and activity (Guo and Cech, 2002). Mutation-induced switches in RNA folding patterns have also been proposed as important events in natural evolution (Falmm et al, 2001), and potentially influence the stability and assembly of the genomes of RNA viruses such as Harvey Sarcoma virus (Rasmussen et al, 2002).

In mammalian cells, mRNA stability is often regulated by attachment of proteins to “instability regions” in the 3′ untranslated region of mRNA. For example, CU-rich regions in the mRNA encoding CD40 ligand protein attach a protein which stabilises the RNA-stability is reduced if this region is mutated (Kosinski et al, 2003). Furthermore, the θ-globin gene shows reduced expression due to ineffective RNA processing as a result of a naturally occurring deletion mutant in the 3′ untranslated region of the gene (Bilenoglu et al, 2002).

By contrast, many cytokine and receptor genes contain an instability sequence AUUUA in the 3′ untranslated region of the mRNA, and mutation or removal of this sequence increases RNA stability and gene expression (Stoecklin et al, 2001; Schaaf and Cidlowski, 2002). Similarly the mRNA from Drosophila melanogaster encoding the ftz protein contains 3 elements that confer instability on the mRNA. Interestingly, while one of these is in the 3′ untranslated region of the RNA, the other two fall within the coding region. Changes to these elements result in increased RNA stability and protein expression (Ito and Jacobs-Lorena, 2001).

In bacterial systems, mRNAs are degraded by “degradosomes” involving the action of an exonuclease such as RNAse E from the 3′ end of the molecule. As in mammalian cells, removal of instability sequences can result in enhanced expression of the protein encoded by the mRNA (Leroy et al, 2002; Cisneros et al, 1996).

Other features of mRNA molecules in addition to stability influence their activity in driving gene expression. These can include silent base changes that affect codon usage without altering the protein sequence, and mutation to a codon for which tRNA is more abundant in the expressing organism may increase the level of protein expression (Widersten et al, 1996; Sutiphong et al, 1987; Sharp and Li, 1986). Mutations which change the coding sequence of the protein may also influence the ultimate level of protein expression, presumably due to increased stability of the product, while mutations that affect RNA secondary structure can alter protein expression by altering the ease of access of the translation machinery to translation initiation sequences. (Sutiphong et al, 1987).

Thus, many features of mRNA molecules interact in determining the level at which an encoded protein is made and can be isolated from the expression system. Similarly many aspects interact in determining the biological activity of RNA molecules with non-coding biological functions. Since the precise interactions of these features will vary from one RNA to another, and one biological system to another, it is not yet possible to precisely tailor RNA molecules for optimal biological function, including optimal protein production. There is thus a need for a system that can efficiently produce variants of the starting RNA molecule and allow for selection of RNAs with the most favourable biological properties. In order to achieve optimisation of RNA for the full range of properties, including stability, folding, binding activity or protein expression, it is essential to access the full range of possible variants of the starting molecule, with mutations to be assessed covering all possibilities in both distribution and type. For example a mutation system such as error-prone PCR, which introduces G-C and C-G switches at extremely low levels (EvoGenix Pty Ltd, unpublished results), will fail to reveal many potentially useful changes in RNA properties which might be accessed by a more complete mutagenesis system. An improved process for generating and selecting mutant RNA molecules with desirable properties is therefore needed.

### RNA-directed RNA Polymerases

Qβ bacteriophage is an RNA phage that infects E. coli. It has an efficient replicase (RNA-dependent RNA polymerases are termed replicases or synthetases) for replicating its single-strand RNA genome of coliphage Qβ. Qβ replicase is error-prone and introduces mutations into the RNA calculated in vivo to occur at a rate of one mutation in every 10³-10⁴ bases. The fidelity of Qβ replicase is low and strongly biased to replicating its template (Rohde et al, 1995). These teachings indicate that replication over a prolonged period leads to accumulation of mutated strands not suitable for synthesis of a desired protein. Both + and −strands serve as templates for replicase; however, for the viral genome the +strand is bound by Qβ replicase and used as the template for the complementary strand (−). In order for RNA replication to occur the replicase requires specific RNA sequence/structural elements which have been well defined (Brown and Gold 1995; Brown and Gold 1996). A reaction containing 0.14 femtograms of a small recombinant RNA has been reported to be amplified by Qβ replicase to 129 nanograms in 30 mins (Lizardi et al, 1988).

RNA-directed RNA polymerases are known to replicate RNA exponentially on compatible templates. Compatible templates are RNA molecules with secondary structure such as that seen in MDV-1 RNA (Nishihara et al, 1983). In this regard, a vector has been described for constructing amplifiable mRNAs as it possesses the sequences and secondary structure (MDV-1 RNA) required for replication and is replicated in vitro in the same manner as Qβ genomic RNA. The MDV-1 RNA sequence (a naturally occurring template for Qβ replicase) is one of a number of natural templates compatible with amplification of RNA by Qβ replicase (U.S. Pat. No. 4,786,600); it possesses tRNA-like structures at its terminus which are similar to structures that occur at the ends of most phage RNAs which increase the stability of embedded mRNA sequences. Linearization of the plasmid allows it to act as a template for the synthesis of further recombinant MDV-1 RNA (Lizardi et al, 1988). Teachings in the art show that prolonged replication by Qβ replicase of a foreign gene requires that it be embedded as RNA within one of the naturally occurring templates for Qβ such as MDV-1 RNA.

### Evolution of Proteins

In vitro evolution of proteins involves introducing mutations into known gene sequences to produce a library of mutant sequences, translating the sequences to produce mutant proteins and then selecting mutant proteins with the desired properties. This process has the potential for generating proteins with improved diagnostic, therapeutic or industrial utility. Unfortunately, however, the potential of this process has been limited by the range of methods available to introduce mutations randomly but with controllable mutation frequency. Some of the most common methods used for mutagenesis include direct replacement, error-prone PCR, RNA replicases, and recombination which can result in mutations at points of rejoining of DNA fragments.

One effective method for in vitro evolution which has recently been described is the use of RNA replicating enzymes to introduce mutations into RNA copies of genes of interest. These enzymes have been demonstrated to introduce errors as they replicate RNA because they lack editing functions (WO 99/58661). While this method is effective in generating variant RNA copies, there are some disadvantages in using this process. For instance, these enzymes require RNA templates and the enzymes can be difficult to obtain.

Other approaches to mutagenesis of nucleic acids are also hampered by difficulties—some introduce mutations in clusters or “hot spots” rather than randomly along the nucleic acid molecule, while others are difficult to control and may introduce an excess of mutations with a resulting loss in utility of the mutated nucleic acid molecule produced. For these reasons the present inventors sought alternative approaches to introducing mutations into nucleic acid molecules.

### SUMMARY OF THE INVENTION

The present inventors have developed a mutagenesis method that can be applied to both RNA and DNA whereby one or more mutations can be introduced during replication or transcription of a target nucleic acid molecule by inclusion of ribavirin, or an analogue or derivative thereof. The method can be used to produce RNA or DNA molecules with improved functionality including enhanced stability or expression of encoded proteins, and as well as nucleic acid molecules encoding proteins with improved activities or properties.

This method is based on the surprising finding that ribavirin is an effective mutagen when used in combination with any one of a range of different polymerases during replication or transcription of RNA or DNA, and that an intact cell is not required for the introduction of mutations. The present inventors have also found that ribavirin can be used to introduce mutations at a relatively low level and thereby effect limited changes to the resulting RNA or DNA molecules. Ribavirin is known to be an effective antiviral agent in the treatment of viruses that have an RNA intermediate as part of their replication cycle. This is thought to be due to the introduction of a high level of mutations which leads to viral death. Ribavirin has not previously been considered for use in introducing desirable mutations during evolution of DNA or RNA molecules. The surprising finding by the inventors that ribavirin can be used under appropriate conditions to introduce mutations at a relatively low level indicates that it is particularly suitable for this purpose.

The present inventors have also recognised that mutagenesis methods using error prone RNA dependent RNA polymerases can be used to produce mutant RNA molecules, which molecules can be selected or used on the basis of an improved functionality of the RNA molecule per se rather than necessarily on the basis of an improved property of their encoded protein.

Accordingly, in a first aspect the present invention provides a method of introducing one or more mutations during replication or transcription of a target nucleic acid molecule, the method comprising

(i) incubating the target nucleic acid molecule with a polymerase in the presence of ribavirin, or a derivative/analogue thereof, under conditions that allow the introduction of a mutation(s) during transcription or replication of the target nucleic acid, and

(ii) selecting a mutant target nucleic acid molecule or selecting for an effect of the introduced mutation(s).

The method of the first aspect invention may be performed in, for example, an in vitro system containing purified components, or in a cell-free system (derived from eukaryotic or prokaryotic sources) containing crude components of unknown composition.

As the skilled addressee would be aware, the method will be performed under any conditions that allow nucleic acid transcription and/or replication. In one embodiment, the method is performed in a cell-free system such as, but not limited to, rabbit reticulocyte lysate, wheatgerm, or E. coli lysate.

Alternatively, the method may be performed in vivo, in yeast, bacterial, mammalian, plant or other cells which replicate and/or transcribe nucleic acids by enzymes other than RNA-dependent RNA polymerases. In a preferred embodiment the cell is not infected with a virus.

In one embodiment the present invention provides a method of introducing one or more mutations during replication or transcription of a target nucleic acid molecule, the method comprising incubating the nucleic acid molecule with a polymerase and nucleosides in the presence of ribavirin, or a derivative/analogue thereof, under conditions which allow transcription or replication of the target nucleic acid, wherein the polymerase is not an RNA dependent RNA polymerase.

The method of first aspect can be used to produce a nucleic acid molecule with an altered phenotype or desired activity. For example, the method of the first aspect can be used to produce a mutant RNA or DNA molecule that exhibits enhanced stability or enhanced levels of expression of a polypeptide. In another example, the method of the first aspect can be used to produce a mutant RNA or DNA molecule where the mutation occurs in a regulatory element, such as an enhancer or a promoter or a fragment thereof, and the RNA or DNA molecule exhibits an altered regulatory activity. In another example, the target nucleic acid is a catalytic molecule, such as a ribozyme or a DNAzyme, and the method is used to produce a mutant molecule exhibiting an altered catalytic activity.

The altered phenotype can also be an altered activity of a protein encoded by the nucleic acid. The altered activity may be a new function that is not possessed by the protein encoded by the nucleic acid before mutation, or an altered level of activity of an existing function.

The method of the first aspect can be adapted in numerous ways to introduce mutations into a nucleic acid molecule. Following the introduction of a mutation(s), the nucleic acid can be copied or amplified (in the absence or presence of further ribavirin or a derivative/analogue thereof), analysed for an altered phenotype (desired activity), or analysed for the ability to encode a protein with an altered phenotype. Further copying or amplifying steps may comprise converting the nucleic acid from DNA to RNA or vice versa. If the mutated nucleic acid is DNA, it will need to be transcribed into RNA before a protein encoded by the DNA can be produced.

In a second aspect, the present invention provides method of identifying a mutant protein with a desired property, the method comprising

(i) incubating a target nucleic acid molecule with a polymerase in the presence of ribavirin, or a derivative/analogue thereof, under conditions which allow transcription or replication of the target nucleic acid,

(ii) producing a protein encoded by a nucleic acid produced from step (i), and

(iii) screening the protein for a desired property.

The method of the second aspect of the invention may be performed in an in vitro system containing purified components, or in a cell-free system (derived from eukaryotic or prokaryotic sources) containing crude components of unknown composition. As the skilled addressee would be aware, the method will be performed under any conditions that allow nucleic acid transcription and/or replication. In one embodiment, the method is performed in a cell-free system such as, but not limited to, rabbit reticulocyte lysate, wheatgerm, or E. Coli lysate.

Alternatively, the method or the second aspect may be performed in vivo, in yeast, bacterial, mammalian, plant or other cells which replicate and/or transcribe nucleic acids by enzymes other than RNA-dependent RNA polymerases. In a preferred embodiment the cell is not infected with a virus.

In yet a further embodiment of the second aspect, the nucleic acid produced from step (i) is copied in the absence of ribavirin or a derivative/analogue thereof before the production of the encoded protein.

In yet another embodiment of the second aspect, the nucleic acid produced from step (i) or a copy thereof is cloned into a suitable vector and transformed/transfected into a host cell before the protein is produced.

In yet a further embodiment of the second aspect, the nucleic acid produced from step (i) is RNA and the method further comprises reverse transcribing the RNA and isolating the resulting DNA before the protein is produced. The DNA may be transformed/transfected into a host cell before the protein is produced.

In one embodiment of the second aspect, the protein is associated with its encoding nucleic acid molecule.

The phrase “associated with,” as used herein, is intended to refer to an association between the translated protein and its corresponding nucleic acid molecule, where the association is maintained through the processes of translation and selection, such that the RNA or corresponding DNA encoding the selected protein can be recovered. The translated protein and its encoding RNA or DNA can be associated with one another via a number of suitable means.

In one particular embodiment, the translated protein and encoding RNA molecule are associated by way of intact ternary ribosome complexes. A ribosome complex preferably comprises at least one ribosome, at least one RNA molecule and at least one translated polypeptide. This complex allows “ribosome display” of the translated protein. Conditions which are suitable for maintaining ternary ribosome complexes intact following translation are known. For example, deletion or omission of the translation stop codon from the 3′ end of the coding sequence results in the maintenance of an intact ternary ribosome complex. Sparsomycin or similar compounds can be added to prevent dissociation of the ribosome complex. Maintaining specific concentrations of magnesium salts and lowering GTP levels may also contribute to maintenance of the intact ribosome complex.

In a further embodiment, the association is facilitated through an RNA binding molecule. In this embodiment, the encoding RNA comprises a sequence encoding the protein of interest, a sequence encoding an RNA binding molecule, and a sequence that may be bound by the de novo translated RNA binding molecule (e.g. an RNA binding motif or domain). The RNA binding molecule may be an RNA binding protein. An example of a suitable RNA binding protein is the coat protein of phage MS2 that forms a complex with a TR 19-nt RNA hairpin structure (replicase translational operator). See, for example, Helgstrand et al 2002. Another example of an RNA binding protein is the VP1 protein of Infectious Bursal Disease Virus (IBDV). The VP1 protein of IBDV is encoded by an RNA sequence to which it will bind. Accordingly, if the encoding RNA includes a coding sequence for VP1, the translated VP1 protein will bind to its own RNA sequence and hold together the quaternary ribosome complex.

In still another embodiment, the translated protein is fused to its encoding RNA. mRNA-protein fusions are described in Roberts (1999). A covalent linkage between mRNA and a translated protein may be formed, for example, by puromycin as described by Nemoto et al (1997) and Roberts and Szostak (1997).

Alternatively, proteins may be “associated” with their encoding nucleic acid molecules by virtue of association with or location within the same cell or viral particle. Preferably, the translated protein is “associated with” the same cell or viral particle as its encoding DNA (or RNA) by, for example, being expressed on the surface of that cell or viral particle.

In a further embodiment of the second aspect, steps (i) and (ii) are carried out simultaneously in either a single or multiple chambered vessel, wherein the multiple chambered vessel allows the transfer of fluids between chambers.

Preferably, the protein is produced in a translation system comprising oxidised and/or reduced glutathione at a total concentration of between about 0.1 mM to about 10 mM. More preferably, the glutathione concentration is between about 2 mM to about 7 mM. Even more preferably, the translation system comprises oxidised glutathione at a concentration of about 2 mM and reduced glutathione at a concentration of between about 0.5 mM to about 5 mM.

In another embodiment of the second aspect, the method further comprises the step of recovering the encoding nucleic acid molecule. The encoding nucleic acid molecule may be recovered by reverse transcription, RT-PCR amplification or PCR amplification.

In one embodiment of the second aspect, the method comprises:

(a) incubating a target DNA molecule with a DNA dependent RNA polymerase in the presence of ribavirin, or a derivative/analogue thereof, under conditions which allow transcription of the target DNA molecule, thereby producing mutant RNA molecules,

(b) producing proteins encoded by mutant RNA molecules produced from step (a), and

(c) screening the proteins for a desired activity.

In another embodiment of the second aspect, the method comprises:

(a) incubating a target DNA molecule with a DNA dependent DNA polymerase in the presence of ribavirin, or a derivative/analogue thereof, under conditions which allow replication of the target DNA molecule, thereby producing mutant DNA molecules,

(b) producing proteins encoded by mutant DNA molecules produced from step (a), and

(c) screening the proteins for a desired activity.

In another embodiment of the second aspect, the method comprises:

(a) incubating a replicable RNA molecule with an RNA dependent RNA polymerase in the presence of ribavirin, or a derivative/analogue thereof, under conditions which allow replication of the RNA molecule, thereby producing mutant RNA molecules,

(b) producing proteins encoded by mutant RNA molecules produced from step (a), and

(c) screening the proteins for a desired activity.

In another embodiment of the second aspect, the method comprises:

(a) incubating an RNA molecule with an RNA dependent DNA polymerase in the presence of ribavirin, or a derivative/analogue thereof, under conditions which allow reverse transcription of the RNA molecule, thereby producing mutant DNA molecules,

(b) producing proteins encoded by mutant DNA molecules produced from step (a), and

(c) screening the proteins for a desired activity.

In another embodiment of the second aspect, the method comprises:

(a) transcribing RNA from a DNA template using a DNA dependent RNA polymerase in the presence of ribavirin, or a derivative/analogue thereof, thereby producing mutant RNA molecules,

(b) incubating mutant RNA molecules produced in step (a) with a translation system under conditions which result in the synthesis of a population of mutant proteins such that after translation, mutant proteins are associated with their encoding RNA molecules;

(c) screening the mutant proteins for a desired activity, and

(d) optionally recovering the encoding RNA molecule.

In another embodiment of the second aspect, the method comprises:

(a) incubating a replicable RNA molecule with an RNA dependent RNA polymerase in the presence of ribavirin, or a derivative/analogue thereof, under conditions which allow replication of the RNA molecule, thereby producing mutant RNA molecules,

(b) incubating mutant RNA molecules produced in step (a) with a translation system under conditions which result in the synthesis of a population of mutant proteins such that after translation, mutant proteins are associated with their encoding RNA molecules;

(c) screening the mutant proteins for a desired activity, and

(d) optionally recovering the encoding RNA molecule.

In another embodiment of the second aspect, the method comprises:

(a) transcribing RNA from a DNA template using a DNA dependent RNA polymerase in the presence of ribavirin, or a derivative/analogue thereof, thereby producing mutant RNA molecules,

(b) reverse transcribing the mutant RNA molecules produced in step (a) thereby producing corresponding mutant DNA molecules;

(c) exposing mutant DNA molecules produced in step (b) to a transcription/translation system under conditions which result in the synthesis of a population of mutant proteins such that after translation, mutant proteins are associated with their encoding DNA molecules;

(d) screening the mutant proteins for a desired activity, and

(e) optionally recovering the encoding DNA molecule.

In another embodiment of the second aspect, the method comprises:

(a) incubating a replicable RNA molecule with an RNA dependent RNA polymerase in the presence of ribavirin, or a derivative/analogue thereof, under conditions which allow replication of the RNA molecule, thereby producing mutant RNA molecules,

(b) reverse transcribing the mutant RNA molecules produced in step (a) thereby producing corresponding mutant DNA molecules;

(c) exposing mutant DNA molecules produced in step (b) to a transcription/translation system under conditions which result in the synthesis of a population of mutant proteins such that after translation, mutant proteins are associated with their encoding DNA molecules;

(d) screening the mutant proteins for a desired activity, and

(e) optionally recovering the encoding DNA molecule.

In a further embodiment of the first and second aspects, the polymerase has an inherently high mutation rate, generally through reduced or deficient proof reading activity. However, the present invention also encompasses the use of polymerases with low error rates, such as T7 RNA polymerase, whilst still ensuring the incorporation of mutations. Advantages being that polymerases with low error rates, such as some DNA dependent RNA polymerases, are typically more readily commercially available, and are significantly cheaper than polymerases which have high mutation rates.

The methods of the first and second aspects may comprise further steps which increase the number of mutations upon transcription. For example, the RNA may be copied by the action of an RNA dependent RNA polymerase which introduces mutations such as, but not limited to, Qβ replicase, Hepatitis C RdRp, Vesicular Stomatitis Virus RdRp, Turnip yellow mosaic virus replicase and RNA bacteriophage phi 6 RNA-dependent RNA polymerase.

The methods of the first and second aspects of present invention may further comprise exposing the target nucleic acid to at least one other mutagen, apart from ribavirin or a derivative/analogue thereof, which also introduce mutations during replication or transcription, and/or procedures which promote mutagenesis. Such other mutagens/mutagenesis procedures may be used, for example, to increase the total number of mutations introduced into the target nucleic acid molecule. These other mutagens/mutagenesis procedures may be utilized before, during or after performing the replication or transcription steps of the invention in the presence of ribavirin or a derivative/analogue thereof. Accordingly, in a preferred embodiment replication or transcription is performed in the presence of at least one other mutagen, preferably a chemical mutagen.

In the context of the first and second aspects of the invention, any process of selecting a mutant protein of interest can be used. For example, selection can be achieved by binding to a target molecule or by measurement of a biological response affected by the mutant protein.

For example, if the protein of interest is an enzyme, the selection process can involve exposing mutant proteins to a target molecule, such as an enzyme substrate, and monitoring the enzymatic activity of the mutant proteins. The enzymatic activity can be monitored, for example, by analyzing whole cells or cell extracts comprising the mutant proteins.

In another example, if the protein of interest is an agent that promotes or reduces cell growth or division, the selection process can involve exposing mutant proteins to a population of cells and monitoring the biological responses of those cells.

In another example, if the mutant protein is a receptor ligand, the process can involve exposing mutant proteins to cells expressing the receptor and monitoring a biological response effected by signalling of the receptor.

In a preferred embodiment, the desired activity is the ability to bind to a target molecule. Examples of a target molecule include, but are not limited to, a DNA molecule, a protein, a receptor, a cell surface molecule, a metabolite, an antibody, a hormone, a bacterium or a virus.

Preferably, the target molecule is bound to a matrix. Furthermore, it is preferred that the matrix comprises magnetic beads.

In one embodiment of the first and second aspects, the polymerase is a DNA dependent RNA polymerase and the target nucleic acid molecule is a DNA molecule. The DNA dependent RNA polymerase can be any such molecule known in the art. Preferred DNA dependent RNA polymerases include, but are not limited to, T7 RNA polymerase, SP6 RNA polymerase and T3 RNA polymerase.

In another embodiment of the first and second aspects, the polymerase is a DNA dependent DNA polymerase and the target nucleic acid molecule is a DNA molecule. Examples include, but are not limited to, Tth DNA polymerase, Vent DNA polymerase, Pwo polymerase, DNA polymerase I Klenow fragment from bacteria such as E. coli, and T4 DNA polymerase.

In a further embodiment of the first and second aspects, the polymerase is a RNA dependent DNA polymerase and the target nucleic acid molecule is a RNA molecule. Examples include, but are not limited to, AMV reverse transcriptase and M-MLV reverse transcriptase, SuperScript III and Tth polymerase.

In yet a further embodiment of the first and second aspects, the polymerase is an RNA dependent RNA polymerase and the target nucleic acid molecule is a RNA molecule. Examples include, but are not limited to, Qβ replicase, Hepatitis C RdRp, Vesicular Stomatitis Virus RdRp, Turnip yellow mosaic virus replicase and RNA bacteriophage phi 6 RNA-dependent RNA polymerase.

The methods of the present invention may further comprise adding nucleic acid precursors, such as nucleosides or nucleotides, prior to or during incubation of the target nucleic acid molecule with the polymerase. Preferably, the precursors are provided as triphosphates (namely nucleotide triphosphates). However, nucleosides/nucleotides may be provided in a non-phosphorylated, mono-phosphate or di-phosphate form and converted to the tri-phosphorylated form by enzymes present in the in vitro system, the cell-free system or within a living cell. When RNA is produced by the transcription or replication procedure the nucleotides provided will preferably be the ribonucleoside triphosphates rATP, rCTP, rGTP and rUTP. When DNA is produced by the transcription or replication procedure the nucleotides provided will preferably be the deoxyribonucleoside triphosphates dATP, dCTP, dGTP and dTTP.

In a third aspect, the present invention provides a kit comprising ribavirin, or a derivative/analogue thereof, and at least one reagent required for the replication or transcription of a nucleic acid molecule.

Preferably, the at least one reagent is selected from the group consisting of a polymerase or a nucleic acid molecule encoding a polymerase, a reaction buffer, and nucleosides or nucleotides.

Preferably, the polymerase has reduced or deficient proof reading activity. Preferably, the polymerase which has reduced or deficient proof reading activity produces, on average, at least 0.05 mutations per 1000 bp duplicated, more preferably at least 0.075 mutations per 1000 bp duplicated, more preferably at least 0.1 mutations per 1000 bp duplicated, more preferably at least 0.2 mutations per 1000 bp duplicated, and even more preferably at least 0.4 mutations per 1000 bp duplicated.

The kit may also comprise a control nucleic acid template. Following instructions provided with the kit the skilled addressee should expect a specified quantity of mutations upon transcription or replication of the control nucleic acid template in the presence of ribavirin or a derivative/analogue thereof. If the specific quantity of mutations is not observed this will indicate that the method is not being performed correctly. Naturally, this enables the skilled addressee to perform routine experimentation to ensure the kit is being used to its optimal potential.

Preferably, the kit further comprises a mutagen, apart from ribavirin or a derivative/analogue thereof.

In a further aspect, the present invention provides a kit comprising ribavirin, or a derivative/analogue thereof, and at least one other mutagen. Preferably, the other mutagen is a chemical mutagen. Examples of suitable mutagens include, but are not limited to, i) mutagens such as sodium bisulfite, nitrous acid, hydroxylamine, hydrazine or formic acid, ii) other analogues of nucleotide/nucleoside precursors such as nitrosoguanidine, 5-bromouracil, 2-aminopurine, 5-formyl uridine, isoguanosine or acridine as well as derivatives/analogues thereof, and iii) intercalating agents such as proflavine, acriflavine and quinacrine.

Preferably, the ribavirin, or derivative/analogue thereof, is provided as a mono- di- or tri-phosphate, however, in at least some embodiments the ribavirin, or derivative/analogue thereof, is converted to the phosphorylated form by enzymes present in the in vitro system, the cell-free system or within a living cell.

Preferably, the concentration of ribavirin, or derivative/analogue thereof, used in the methods of the invention is between about 10 μM and about 20 mM, more preferably between about 100 μM and about 10 mM, even more preferably between about 500 μM and about 5 mM. In one embodiment, the concentration of ribavirin, or derivative/analogue thereof, is about 1000 μM. In another embodiment, the concentration of ribavirin, or derivative/analogue thereof, is about 2000 μM.

In a fourth aspect the present invention provides method for identifying a mutant RNA molecule which exhibits an altered property or activity, the method comprising

(i) incubating a target RNA molecule with an RNA dependent RNA polymerase under conditions wherein the RNA dependent RNA polymerase replicates the RNA molecule but introduces a mutation(s) thereby generating a population of mutant RNA molecules; and

(ii) selecting a mutant RNA molecule that exhibits an altered property or activity.

In one embodiment of the fourth aspect the altered property or activity is enhanced expression of an encoded polypeptide when compared to the level of expression of the polypeptide before the introduction of a mutation(s) in step (i).

In another embodiment of the fourth aspect the altered property or activity is enhanced stability when compared to the level of stability before the introduction of a mutation(s) in step (i).

In another embodiment of the fourth aspect the altered property or activity is altered catalytic activity when compared to the level of catalytic activity before the introduction of a mutation(s) in step (i).

In another embodiment of the fourth aspect the altered property or activity is enhanced RNA interference activity when compared to the level of RNA interference activity before the introduction of a mutation(s) in step (i).

In another embodiment of the fourth aspect the altered property or activity is enhanced antisense activity when compared to the level of antisense activity before the introduction of a mutation(s) in step (i).

In another embodiment of the fourth aspect the mutations introduced into the RNA molecule in step (i) do not alter the amino acid sequence of a protein encoded by the RNA molecule.

A number of RNA-directed RNA polymerases (otherwise known as replicases or RNA synthetases) known in the art have been isolated and are suitable for use in the method of the fourth aspect. Examples of these include bacteriophage RNA polymerases, plant virus RNA polymerases and animal virus RNA polymerases. In a preferred embodiment of the present invention, the RNA-directed RNA polymerase introduces mutations into the replicated RNA molecule at a relatively high frequency, preferably at a frequency of at least one mutation in 10⁴ bases, more preferably one mutation in 10³ bases. In a more preferred embodiment the RNA-directed RNA polymerase is selected from the group consisting of Qβ replicase, Hepatitis C RdRp, Vesicular Stomatitis Virus RdRp, Turnip yellow mosaic virus replicase (Deiman et al, 1997) and RNA bacteriophage phi 6 RNA-dependent RNA polymerase (Ojala and Bamford, 1995). Most preferably, the RNA-directed RNA polymerase is Qβ replicase.

The RNA-directed RNA polymerase can be included in the transcription/translation system as a purified protein. Alternatively, the RNA-directed RNA polymerase can be included in the form of a gene template which is expressed during replication of the RNA molecule.

In a further preferred embodiment, the RNA-directed RNA polymerase can be fused with or associated with a target molecule. Without wishing to be bound by theory, it is envisaged that in some cases, the binding affinity of the translated protein for the target can be greater than the affinity of the replicase for the RNA molecule. The binding of the mutant protein/RNA complex to a target molecule/RNA-directed RNA polymerase fusion construct would bring the RNA into the proximity of the RNA-directed RNA polymerase. This may result in preferential further replication and mutation of RNA molecules of interest.

RNA templates that are replicated by various RNA-dependent RNA polymerases are known in the art and may serve as vectors for producing replicable RNAs suitable for use in the present invention. Known templates for Qβ replicase include RQ135 RNA, MDV-1 RNA, microvariant RNA, nanovariant RNAs, CT-RNA and RQ120 RNA. Qβ RNA, which is also replicated by Qβ replicase, is not preferred, because it has cistrons, and further because the products of those cistrons regulate protein synthesis. Preferred vectors include MDV-1 RNA (Kramer et al, 1978) and RQ135 RNA (Munishkin et al, 1991) (RQ135). They can be made in DNA form by well-known DNA synthesis techniques.

In a preferred embodiment, the method further includes the step of transcribing a DNA construct to produce replicable RNA. DNA encoding the recombinant RNA can be, but need not be, in the form of a plasmid. It is preferable to use a plasmid and an endonuclease that cleaves the plasmid at or near the end of the sequence that encodes the replicable RNA in which the gene sequence is embedded. Linearization can be performed separately or can be coupled with transcription-replication-translation. Preferably, however, linear DNA is generated by any one of the many available DNA replication reactions and most preferably by the technique of Polymerase Chain Reaction (PCR). For some systems non-linearized plasmids without endonuclease may be preferred. Suitable plasmids can be prepared, for example, by following the teachings of Melton et al (1984a, b) regarding processes for generating RNA by transcription in vitro of recombinant plasmids by bacteriophage RNA polymerases, such as T7 RNA polymerase or SP6 RNA polymerase (Melton et al, 1984a and 1984b). It is preferred that transcription begin with the first nucleotide of the sequence encoding the replicable RNA.

Step (i) and/or step (ii) of the method of the fourth aspect of the invention may be performed in an in vitro system containing purified components, or in a cell-free system (derived from eukaryotic or prokaryotic sources) containing crude components of unknown composition. In one embodiment, the method is performed in a cell-free system such as, but not limited to, rabbit reticulocyte lysate, wheatgerm, or E. coli lysate.

Alternatively, step (i) and/or step (ii) of the method of the fourth aspect of the invention may be performed within a cell.

As the above-mentioned aspects of the invention relate to methods of introducing mutations into a target RNA molecules, procedures known to enhance mutagenesis can be used in conjunction with these methods. Thus, the method of the fourth aspect may further comprise exposing the target nucleic acid to other mutagens, such as ribavirin or a derivative/analogue thereof, which also introduce mutations during replication or transcription, and/or procedures which promote mutagenesis. Such other mutagens can be used to increase the total number of mutations introduced into the target RNA molecule. In a preferred embodiment replication is performed in the presence of at least one chemical mutagen.

Following the introduction of the mutation(s), the mutant RNA population can be copied or amplified and analysed for an altered phenotype (desired activity). Further copying or amplifying steps may comprise converting the nucleic acid from RNA to DNA.

It will be appreciated that RNA and DNA molecules produced by methods of the present invention will be particularly advantageous as therapeutic or prophylactic agents. For example, RNA and DNA molecules that exhibit enhanced stability or enhanced expression of the encoded polypeptide will be particularly useful in methods of gene therapy or in nucleic acid vaccine compositions. Catalytic RNA molecules, dsRNA molecules and antisense constructs exhibiting enhanced stability or enhanced catalytic or antisense activity will also be particularly advantageous therapeutic agents.

Accordingly, in one further embodiment of the invention, RNA which encodes a protein of interest for use as a vaccine component or for gene therapy is mutated by any of the methods of the invention and selected for an improved stability to potential inactivating entities including nucleases. This stabilized RNA will be administered directly to a patient in need of vaccination or gene therapy, by any of the many known techniques for such administration. Such stabilised RNA can be expected to express its encoded protein over a useful but finite time period. The problems of indefinite long term expression and potential incorporation into the host cell genome associated with DNA administration would be avoided by the use of the stabilised RNA of the invention.

In another aspect, the present invention provides a mutant nucleic acid, and/or mutant protein encoded thereby, produced using a method of the invention. Also provided is a composition comprising a mutant nucleic acid, and/or mutant protein encoded thereby, produced using a method of the invention, for use in medical, agricultural or industrial purposes.

As will be apparent, preferred features and characteristics of one aspect of the invention are applicable to many other aspects of the invention.

The terms “comprise”, “comprises” and “comprising” as used throughout the specification are intended to refer to the inclusion of a stated component or feature or group of components or features with or without the inclusion of a further component or feature or group of components or features.

### BRIEF DESCRIPTION OF THE DRAWINGS

Various preferred features and embodiments of the present invention will now be described in more detail by way of non-limiting example and with reference to the accompanying Figures, in which:

FIG. 1 shows plasmid pEGX207. The base plasmid used for construction of pEGX207 was pUC18 with a T7 RNA promoter and RQ-EGX sequence inserted at the multi-cloning site of pUC18 between the PstI and SmaI restriction sites. The T7 RNA promoter sequence is followed by an RQ135 sequence to permit amplification of RNA by Qb polymerase.

FIG. 2 shows the predicted structure of an RNA molecule encoding a binding protein as generated by a computer program (RNAdraw v1.1). FIG. 2a shows the predicted structure for the wild-type RNA molecule and FIG. 2b shows the predicted structure for a variant RNA molecule selected following mutagenesis according to the methods of the present invention, for increased expression.

FIG. 3a shows expression analysis of a 12Y-2 variant protein (encoded by pEGX248) compared to wild-type 12Y-2. FIG. 3b shows purification of the 12Y-2 variant protein (encoded by pEGX248) compared to wild-type 12Y-2.

FIG. 4 shows the number of mutations in the dihydrofolate reductase gene (DHFR) generated by T7 polymerase versus T7 polymerase in combination with ribavirin-5′-triphosphate according to the methods of the present invention.

### DETAILED DESCRIPTION

### General Techniques and Definitions

Unless otherwise indicated, the recombinant DNA techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al, Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al (Editors), Current Protocols in Molecular Biology, Greene Pub. Associates and Wiley-Interscience (1988, including all updates until present) and are incorporated herein by reference. In particular, these documents describe in detail methods of transcribing or replicating nucleic acid molecules and suitable conditions required therefor.

“Nucleoside”, as used herein, refers to a compound consisting of a purine [guanine (G) or adenine (A)] or pyrimidine [thymine (T), uridine (U) or cytidine (C)] base covalently linked to a pentose, whereas “nucleotide” refers to a nucleoside phosphorylated at one of its pentose hydroxyl groups. “XTP”, “XDP” and “XMP” are generic designations for ribonucleotides and deoxyribonucleotides, wherein the “TP” stands for triphosphate, “DP” stands for diphosphate, and “MP” stands for monophosphate, in conformity with standard usage in the art. Subgeneric designations for ribonucleotides are “NMP”, “NDP” or “NTP”, and subgeneric designations for deoxyribonucleotides are “dNMP”, “dNDP” or “dNTP”. Also included as “nucleoside”, as used herein, are materials that are commonly used as substitutes for the nucleosides above such as modified forms of these bases (e.g. methyl guanine) or synthetic materials well known in such uses in the art, such as inosine.

### Ribavirin and Derivatives/Analogues Thereof

Ribavirin (1-beta-D-ribofuranosyl-1,2,4-triazole) (Formula I), known by the trade name Virazole (also known as Rebetron in combination with interferon-α), is a broad-spectrum antiviral nucleoside discovered by Sidwell and co-workers in 1972. Ribavirin can be obtained from commercial suppliers (e.g., Sigma and ICN). Ribavirin exhibits antiviral activity against a broad range of viruses in cell culture including RNA viruses from the families of arenaviruses, bunyaviruses, flaviviruses orthomyxoviruses, paramyxoviruses, picornaviruses, reoviruses, and some DNA viruses which replicate via a double stranded RNA intermediate (Markland et al, 2000). The efficacy of ribavirin is limited in animal model systems, generally being effective against a more limited set of RNA viruses only (Durr and Lindh, 1975; Hruska et al, 1982; von Herrath et al, 2000). In humans, ribavirin is currently used to treat severe cases of respiratory syncytial virus (Wyde, 1998) and Lassa fever virus (McCormick et al, 1986) or in combination with interferon-α to treat hepatitis C virus infections (McHutchison et al, 1998).

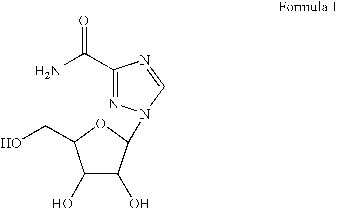

Ribavirin derivatives/analogues useful for the methods of the present invention include, but are not limited to, molecules falling within the generic Formulae II to V, wherein X is O, S, CH₂, CHOH or N—CO—R₁₁; A, B and C are independently N, P, CH, C—OH, C—CH₃, C-alkyl, C-alkenyl, C—CH₂, —CN, C-halogen, C—CN, C—COOCH₃, C—NH₂, C—SNH₂, C—SO₂—NH₂, C—CONH₂, C—CS—NH₂, C—C(NH)NH₂, CPO₂—NH₂, or C-heterocyclic ring system; D is S, Se, Te, PH, NH or NR₁₂; R₁ is H, (CH₂)p(OH), halogen, CN, (CH₂)pONH₂, (CH₂)pNH₂, CH₃, CH₂SPH or (CH₂)-heterocyclic ring; R₂ is H, OH, OCH₃, SH, SCH₃, halogen, CN, NH₂, ONH₂, NHCH₃ (CH₂)OH, (CH₂)pNH₂, CH₃, or COOMe; R₃, R₄, R₅, R₆, R₇ and R₈ are independently H, OH, OCH₃, SH, SCH₃, halogen, CN, NH₂, ONH₂, NHCH₃ (CH₂)OH, (CH₂)pNH₂, CH₃, or COOMe or phenyl; R₉ is H, halogen, NH₂ CH₃, CONH, CSNH₂, COOMe, SNH₂ SO₂NH₂, PO₂NH₂, (CH₂)p, (CH₂)p-heterocyclic ring system or (CH₂)p-glucose; R₁₀ is H, halogen, NH₂, CH₃, CONH, CSNH₂, COOMe, SNH₂, SO₂NH₂, PO₂NH₂, (CH₂)p, (CH₂)p-heterocyclic ring system or (CH₂)p-glucose, O—CH₃, O—CH₂CH₃ or amino acid: Y is O, S, NH.HCI, NOH, NOCH₃ or NOCH₂PH; R₁₀ & Y in combination are a heterocyclic ring systems such as thiazole, imidazole, etc., R₁₁ is CH₃(CH₂)pNH₂, (CH₂)p-heterocycle, (CH₂)p-amino acid or (CH₂)p-sugar (glucose etc); p is an integer between 0 and 8.

These derivatives/analogues can be synthesized by methods known in the art such as those provided in WO 97/26883 and WO 01/45509.

Ribavirin, and derivatives/analogues thereof, may be utilized in non-phosphorylated or phosphorylated forms.

Ribavirin, and derivatives/analogues thereof, can either be in their respective L-configuration or D-configuration. Thus, the L-configuration of ribavirin, namely (1-β-L-ribofuranosyl-1,2,4-triazole-3-carboxamide), which is sold under the trade name “Levovirin” is also useful for the methods of the present invention.

Derivatives/analogues of ribavirin include fatty acid esters. In particular, the fatty acid ester can be a mono-saturated C18 or C20 acid as generally described in U.S. Pat. No. 6,153,594. Such fatty acid esters are especially useful for in vivo or whole cell mutagenesis where the derivative/analogue of ribavirin is required to cross cell membranes.

Ribavirin, and derivatives/analogues thereof, useful for the methods of the present invention can also comprise a 2′-deoxyribose which can be readily incorporated into DNA molecules by DNA polymerases.

In addition, further ribavirin derivatives/analogues can be generated using conventional techniques in rational drug design and combinatorial chemistry. By one approach, the chemical structure of ribavirin is recorded on a computer readable medium and is accessed by one or more modeling software application programs. Compounds having the same structure as the modeled ribavirin derivatives/analogues created in the virtual library are then made using conventional chemistry or can be obtained from a commercial source. The newly manufactured ribavirin derivatives/analogues are then screened for use in the methods of the present invention.

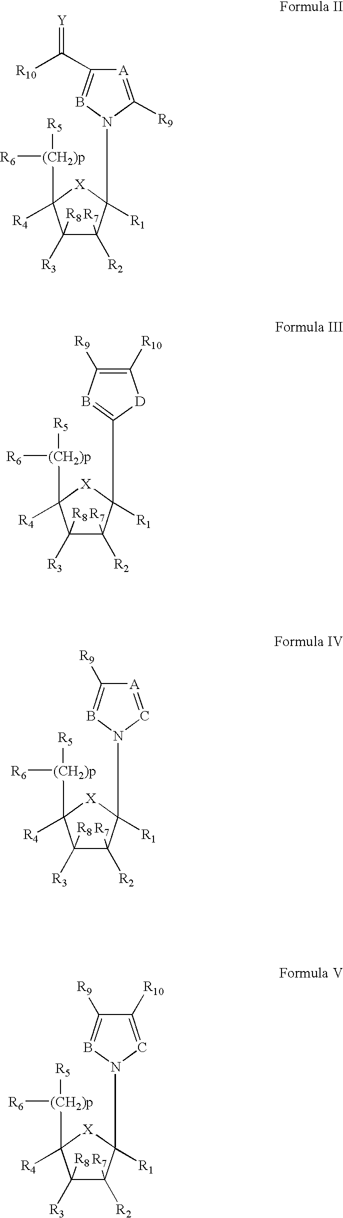

All possible prodrug forms of ribavirin, and derivatives/analogues thereof, are also appropriate for use in the methods of the present invention. Particularly contemplated prodrug forms include, but are not limited to, covalent modifications that may be enzymatically removed from the compounds by the action of enzymes such as aminohydrolases, oxidoreductases or transferases, which may be present in in vivo or cell free systems.

### Target Nucleic Acids and the Transcription/Replication Thereof

The target nucleic acid may be a functional nucleic acid sequence (for example, a regulatory element such as a promoter or enhancer element, a catalytic molecule, a dsRNA or an antisense molecule) or encode a protein of interest. In some circumstances, the target nucleic acid will be unknown. In a preferred embodiment the target nucleic acid encodes i) a library of target binding proteins or ii) a single target binding protein, where the target may include any of a cell surface molecule, receptor, enzyme, antibody or fragment thereof, hormone, a microbe such as a virus, or other molecule or complex or derivative thereof.

The target nucleic acid may also encode a domain which is a tag that is fused or otherwise coupled thereto to assist in purification of an encoded protein. Suitable tag moieties include, for example, a His tag, glutathione-S-transferase (GST), “FLAG” epitope (DYKDDDDK) (SEQ ID NO:1) (International Biotechnologies), or any of the human or murine antibody constant domains. Preferably, the tag is the constant domain from a mouse monoclonal antibody, such as constant domain 1C3. A further preferred tag is the constant region from a human IgM antibody.

The target nucleic acid may further comprise 5′ and 3′ untranslated regions. The 5′ untranslated region will require suitable control elements to promote transcription of the nucleic acid. Since in some embodiments the transcribed RNA will be translated into a protein the nucleic acid template may also comprise a ribosome binding site.

In some circumstances, the template will be DNA which comprises a translation termination (stop) nucleotide sequence. However, in some DNA template constructs, particularly those where encoded proteins are to be examined by ribosome display (see below), it is envisaged that no stop codons should be present to prevent recognition by release factors and subsequent ribosome release. In these circumstances factors such as the antisense ssrA oligonucleotide sequence is added to prevent addition of a C-terminal protease site in the 3′ untranslated region that follows. The addition of sparsomycin, or other similar compounds, or a reduction in temperature also prevents release of the ribosome from the mRNA and de novo synthesised protein.

In other embodiments, the target nucleic acid is mutated and cloned into a suitable expression vector which comprises the necessary regulatory regions for transcription, and optionally translation.

Antisense Compounds

The term “antisense compounds” encompasses DNA or RNA molecules that are complementary to at least a portion of a target mRNA molecule (Izant and Weintraub, 1984; Izant and Weintraub, 1985) and capable of interfering with a post-transcriptional event such as mRNA translation. Antisense oligomers complementary to at least about 15 contiguous nucleotides of the target-encoding mRNA are preferred, since they are easily synthesized and are less likely to cause problems than larger molecules when introduced into the target mRNA producing cell. The use of antisense methods is well known in the art (Marcus-Sakura, 1988).

Catalytic RNA Molecules

The term catalytic RNA refers to an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T and U, as well as derivatives thereof. Derivatives of these bases are well known in the art.

Typically, the catalytic nucleic acid contains an antisense sequence for specific recognition of a target nucleic acid, and a nucleic acid cleaving enzymatic activity (also referred to herein as the “catalytic domain”).

The types of ribozymes that are particularly useful in this invention are the hammerhead ribozyme (Haseloff and Gerlach 1988, Perriman et al, 1992) and the hairpin ribozyme (Shippy et al, 1999).

The ribozymes used in this invention can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette.

dsRNA

dsRNA is particularly useful for specifically inhibiting the production of a particular protein. Although not wishing to be limited by theory, Dougherty and Parks (1995) have provided a model for the mechanism by which dsRNA can be used to reduce protein production. This model was modified and expanded by Waterhouse et al (1998). This technology relies on the presence of dsRNA molecules that contain a sequence that is essentially identical to the mRNA of the gene of interest. Conveniently, the dsRNA can be produced in a single open reading frame in a recombinant vector or host cell, where the sense and anti-sense sequences are flanked by an unrelated sequence which enables the sense and anti-sense sequences to hybridize to form the dsRNA molecule with the un-elated sequence forming a loop structure. The design and production of suitable dsRNA molecules targeted against genes of interest is well within the capacity of a person skilled in the art, particularly considering Dougherty and Parks (1995), Waterhouse et al (1998), WO 99/32619, WO 99/53050, WO 99/49029, and WO 01/34815.

As used herein, the terms “small interfering RNA”, and “RNAi” refer to homologous double stranded RNA (dsRNA) that specifically targets a gene product, thereby resulting in a null or hypomorphic phenotype. Specifically, the dsRNA comprises two nucleotide sequences derived from the target RNA and having self-complementarity such that they can anneal, and interfere with expression of a target gene, presumably at the post-transcriptional level. RNAi molecules are described by Fire et al (1998) and reviewed by Sharp (1999).

### Mutation by Qβ Replicase

Multiple copies of a single-stranded RNA template are generated as a result of the action of Qβ replicase. These copies incorporate mutations and can themselves act as templates for further amplification by Qβ replicase as both RNA strands are equally efficient as templates under isothermal conditions.

Teaching in the art indicates that the complex and stable secondary and tertiary structures present in full length RNA from phages such as Qβ limit the access of ribosomes to the protein initiation sites. However, the present inventors have found that smaller RNA sequences are suitable for binding of replicases and therefore can be used instead of full-length templates. Preferred sequences are small synthetic RNA sequences known as pseudoknots (Brown and Gold 1995; 1996), which are compatible with amplification by Qβ replicase. In the context of the present invention, the use of pseudoknots can overcome the problems of ribosome access to the protein initiation sites whilst maintaining the binding sites necessary and sufficient for the Qβ replicase amplification of the RNA and sequences fused thereto.

### Expression Vectors

Proteins with an altered phenotype can be identified by cloning the nucleic acids obtained using the methods of the invention into suitable host cells and screening the proteins produced by these recombinant cells for the desired activity. Alternatively, a target nucleic acid may be cloned into a suitable vector, this vector subjected to the mutagenesis methods of the invention in cell free systems and the resulting products transformed/transfected into a suitable host cell.

Expression vectors as described herein may be used to transcribe or replicate functional nucleic acids, produced using the methods of the invention, but which are not translated into a protein. Examples of such functional nucleic acids include ribozymes, dsRNA and antisense polynucleotides.

Expression vectors useful in the methods of the invention may be either self-replicating extrachromosomal vectors or vectors which integrate into a host genome. Generally, these expression vectors include transcriptional and translational regulatory nucleic acid operably linked to the nucleic acid encoding the variant protein. The term “control sequence” or grammatical equivalents thereof, as used herein, refer to nucleic acid sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize polyadenylation signals and enhancers.

Nucleic acid is “operably linked” when it is placed into a functional relationship with another nucleic acid sequence. For example, DNA encoding a presequence or secretory leader is operably linked to DNA encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the nucleic acid sequences being linked are contiguous, and, in the case of a secretory leader, contiguous and in reading frame. However, enhancers do not have to be contiguous. Linking can be accomplished by ligation at convenient restriction sites. If such sites do not exist, synthetic oligonucleotide adaptors, linkers or recombination methods are used in accordance with conventional practice. The transcriptional and translational regulatory nucleic acid will generally be appropriate to the host cell used to express the fusion protein; for example, transcriptional and translational regulatory nucleic acid sequences from Aspergillus are preferably used to express the protein in Aspergillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. Regulatory sequences may also include independent nucleic acid molecules that regulate the activity of another gene, for example by influencing RNA splicing. In a preferred embodiment, the regulatory sequences include a promoter and transcriptional start and stop sequences. Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in filamentous fungi cells for expression and in a prokaryotic host for cloning and amplification. Furthermore, for integrating expression vectors, the expression vector can be integrated randomly into the genome or contain at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art. In addition, in a preferred embodiment, the expression vector contains a selectable marker gene to allow the selection of transformed host cells. Selection genes are well known in the art and will vary with the host cell used.

### In Vitro Translation Systems

In the methods of the present invention, the translation of proteins may occur within a cell-free translation system. The translation system can be any such system known in the art, including those derived from prokaryotes or eukaryotes. Examples include the use of rabbit reticulocyte lysates (He and Taussig, 1997) or an E. coli S-30 transcription translation mix (Mattheakis et al, 1994; Zubay, 1973). For mRNA synthesis in eukaryotic cells the mRNA is preferably capped which is achieved by adding an excess of diguanosine triphosphate; however, the rabbit reticulocyte system from the commercial suppliers Promega and Novagen have components in the system to make the addition of capping compounds unnecessary. The coupled transcription/translation system may be extracted from the E. coli mutator cells MUTD5-FIT (Irving et al, 1996) which bear a mutated DNAQ gene and therefore allow further random mutations introduced into DNA during replication as a result of proofreading errors. Addition of glutathione to the coupled system enhances correct folding of displayed proteins and therefore enhances subsequent binding and selection to counter-receptors or antigens.

In addition, there are preferred requirements for the correct folding of the molecules in cell-free in vitro evolution systems. For prokaryotes, protein disulphide isomerase (PDI) and chaperones may be used as well as a C-terminal anchor domain to ensure the correct folding. The latter is required as prokaryotic proteins are released from the ribosomes prior to folding (Ryabova et al, 1997) and therefore in situations in which the peptide is anchored to the ribosome the entire protein needs to be spaced from the ribosome. In contrast to this, in eukaryotic systems the protein is folded as it is synthesised and has no requirement for the prokaryote PDI and chaperones to be added. However, it has been found that addition of a specific range of glutathione concentrations is beneficial to the library selection by the enhanced display of correctly folded proteins on the ternary ribosome complexes.

### In Vivo Translation Systems

In the methods of the present invention, the translation of proteins may occur within whole cells. The nucleic acids are introduced into the cells, either alone or in combination with an expression vector. By “introduced into” or grammatical equivalents herein is meant that the nucleic acids enter the cells in a manner suitable for subsequent expression of the nucleic acid. The method of introduction is largely dictated by the targeted cell type, discussed below. Exemplary methods include PEG mediated protoplast transformation, CaPO₄ precipitation, liposome fusion, Lipofectin™ (e.g., formulation of cationic lipids), electroporation, viral infection, etc. The nucleic acids may stably integrate into the genome of the host cell, or may exist either transiently or stably in the cytoplasm (i.e. through the use of traditional plasmids, utilizing standard regulatory sequences, selection markers, etc.).

Proteins encoded by the mutant nucleic acids produced using the methods of the invention can be produced by culturing a host cell transformed either with an expression vector containing nucleic acid encoding the protein or with the nucleic acid encoding the protein alone, under the appropriate conditions to induce or cause expression of the protein.

The conditions appropriate for protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction.

Appropriate host cells include yeast, bacteria, archaebacteria, fungi, and insect and animal cells, including mammalian cells. Specific examples include, but are not limited to, Drosophila melanogaster and other insect cells, Saccharomyces cerevisiae and other yeasts such as Pichia pastoris, E. coli, Bacillus sp., SF9 cells, C129 cells, 293 cells, Neurospora sp., Trichoderma sp., Aspergillus sp., Fusarium sp., Penicilliuma sp., Streptomyces sp., and mammalian cells such as BHK, CHO, COS, etc.

In one embodiment, the proteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for the fusion protein into mRNA. A promoter will have a transcription initiating region, which is usually placed proximal to the 5′ end of the coding sequence, and a TATA box, usually located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40.

The methods of introducing exogenous nucleic acid into mammalian hosts, as well as other hosts, are well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the nucleic acid into nuclei.

As will be appreciated by those skilled in the art, the type of mammalian cells used in the present invention can vary widely. Basically, any mammalian cells may be used, with mouse, rat, hamster, primate and human cells being particularly preferred, although as will be appreciated by those in the art, modifications of the system by pseudotyping allows all eukaryotic cells to be used, preferably higher eukaryotes. Accordingly, suitable mammalian cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, COS, etc (see the ATCC cell line catalog, hereby expressly incorporated by reference).

In one embodiment, the cells may be additionally genetically engineered, that is, they contain exogenous nucleic acid other than the recombined nucleic acid produced using the methods of the present invention.

In a preferred embodiment, the proteins are expressed in bacterial systems. Bacterial expression systems are well known in the art. A suitable bacterial promoter is any nucleic acid sequence capable of binding bacterial RNA polymerase and initiating the downstream (3′) transcription of the coding sequence of the protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the expressed protein in bacteria. The signal sequence typically encodes a signal peptide comprised of hydrophobic amino acids, which direct the secretion of the protein from the cell, as is well known in the art. The protein can be secreted into the growth media (gram-positive bacteria) or into the periplasmic space, located between the inner and outer membrane of the cell (gram-negative bacteria). The expressed protein may also be accumulated within inclusion bodies within a bacterial cell wall. For expression in bacteria, usually bacterial secretory leader sequences, operably linked to the recombined nucleic acid, are preferred.

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others.

The bacterial expression vectors are transformed into bacterial host cells using techniques well known in the art, such as calcium chloride treatment, electroporation, and others.

In another embodiment, proteins encoded by nucleic acids obtained using the methods of the invention are produced in insect cells. Expression vectors for the transformation of insect cells, and in particular, baculovirus-based expression vectors, are well known in the art.

In further embodiment, proteins encoded by nucleic acids obtained using the methods of the invention are produced in yeast cells. Yeast expression systems are well known in the art, and include expression vectors for Saccharomyces cerevisiae, Candida albicans and C. maltosa, Hansenula polymorpha, Kluyveromyces fragilis and K lactis, Pichia guillerimondii and P. pastoris, Schizosaccharomyces pombe, and Yarrowia lipolytica. Preferred promoter sequences for expression in yeast include the inducible GAL 1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include URA3, ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

In addition, the proteins encoded by nucleic acids obtained using the methods of the invention may be further fused to other proteins, if desired, for example to increase expression or increase stability.

In a further embodiment, the protein encoded by nucleic acids obtained using the methods of the invention is purified or isolated after expression. The proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the protein may be purified using a standard antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the protein. In some instances no purification may be necessary.

### Use of Additional Mutagenic Processes or Mutagens

The methods of the present invention may further comprise exposing the target nucleic acid to other mutagens, apart from ribavirin or a derivative/analogue thereof, which also introduce mutations during replication or transcription, and/or procedures which promote mutagenesis. Such other mutagens/mutagenesis procedures can be used to increase the total number of mutations introduced into the target nucleic acid molecule. These other mutagens/mutagenesis procedures may be utilized before, during or after performing the replication or transcription steps of the present invention.

There are many factors which are commonly used in the art to increase mutation frequency including, but not limited to, use of polymerases with a high error rate (typically as a result of the polymerase having reduced or deficient proof reading activity), performing the reactions under conditions which increase mutation frequency (error prone PCR), irradiation, DNA shuffling techniques, nucleotide/nucleoside analogues (other than ribavirin or a derivative/analogue thereof), and intercalating agents.

Error-prone PCR uses low-fidelity polymerization conditions to introduce a low level of point mutations randomly over a long sequence (Leung et al, 1989; Caldwell and Joyce, 1992). Error prone PCR generally involves performing a PCR reaction with the addition of varying amounts of manganese and dGTP. DNA dependent DNA polymerases such as Taq polymerase require Mg2+ for activity and fidelity. By adding Mn2+ to the PCR reaction (up to a maximum of 650 uM Mn2+), the fidelity of Taq polymerase decreases and leads to mis-incorporation along the DNA template. This mis-incorporation can be increased further by fixing the Mn2+ concentration at the upper limit and biasing the nucleotide pool with the addition of extra dGTP (from 40 to 300 μM). With these modifications to PCR, the mutation rate can theoretically be adjusted to provide mutation rates from 2 to 8 mutations per 1,000 base pairs dependent on the concentration of Mn2+ and the concentration of dGTP added to the PCR reaction.

Error prone PCR using the Diversify™ PCR random mutagenesis kit from BD Biosciences can be performed as outlined in the Table 1. Each buffer condition incorporated a different concentration of Mn²⁺ and dGTP. The anticipated error rate for each buffer condition is also included in the table and is based on data accumulated by BD Biosciences.

TABLE 1
Reaction components for carrying out error-prone PCR and expected
mutation frequencies. The example given is for amplification and
mutagenesis of the dihydrofolate reductase (DHFR) gene.

| | Buffer Condition | | | | | |
|---|---|---|---|---|---|---|
| | | | | | STD | |
| Reagents (μl) | 1 | 2 | 3 | 4 | PCR^{#} | Control^{@} |
| dH₂O | 48 | 38 | 36 | 32 | 41 | 41 |
| 10X Titanium Taq Buffer | 5 | 5 | 5 | 5 | 5 | 5 |
| 8 mM MnSO₄ | 0 | 2 | 4 | 4 | 0 | 0 |
| 2 mM dGTP | 1 | 1 | 1 | 5 | 0 | 0 |
| 50X Diversify dNTP Mix | 1 | 1 | 1 | 1 | 0 | 0 |
| 50X 10 mM dNTP mix | 0 | 0 | 0 | 0 | 1 | 1 |
| DHFR forward primer | 1 | 1 | 1 | 1 | 1 | 1 |
| (10 μM) | | | | | | |
| DHFR reverse primer | 1 | 1 | 1 | 1 | 1 | 1 |
| (10 μM) | | | | | | |
| DHFR DNA template | 1 | 1 | 1 | 1 | 1 | 0 |
| (1 ng/ul) | | | | | | |
| Titanium Taq polymerase | 1 | 1 | 1 | 1 | 1 | 1 |
| Expected Mutation Rate per | 2 | 2.7 | 4.6 | 8.1 | 0.4 | — |
| 1,000 bp* | | | | | | |

*Expected Mutation Rate per 1,000 bp as stated by BD Biosciences with their Diversify ™ PCR random mutagenesis kit for the buffer condition stated
^{#}Standard (i.e. low error rate) PCR reaction using Titanium Taq polymerase
^{@}Negative control reaction that does not contain DNA template

An example of thermal cycler conditions which can be used is:

1 cycle of:
94° C. for 30 sec

25 cycles of:
94° C. for 30 sec
68° C. for 60 sec

1 cycle of:
68° C. for 60 sec.

Alternatively, further mutations can be introduced into the template polynucleotide by oligonucleotide-directed mutagenesis. In oligonucleotide-directed mutagenesis, a short sequence of the polynucleotide is removed from the polynucleotide using restriction enzyme digestion and is replaced with a synthetic polynucleotide in which various bases have been altered from the original sequence.

DNA shuffling methods rely on the mixing and concatenation of genetic material from a number of parent sequences. There are many variations of this procedure known in the art, see for example, Stemmer, (1994), Volkov and Arnold (2000), USSN 20030194763, and USSN 20030186356.

The polynucleotide sequence can also be altered by chemical mutagenesis. Chemical mutagens include, for example, sodium bisulfite, nitrous acid, hydroxylamine, hydrazine or formic acid. Other agents which are analogues of nucleotide or nucleoside precursors include nitrosoguanidine, 5-bromouracil, 2-aminopurine, 5-formyl uridine, isoguanosine, acridine and of N⁴-aminocytidine, N¹-methyl-N⁴-aminocytidine, 3,N⁴-ethenocytidine, 3-methylcytidine, 5-hydroxycytidine, N⁴-dimethylcytidine, 5-(2-hydroxyethyl)cytidine, 5-chlorocytidine, 5-bromocytidine, N⁴-methyl-N.sup.4-aminocytidine, 5-aminocytidine, 5-nitrosocytidine, 5-(hydroxyalkyl)-cytidine, 5-(thioalkyl)-cytidine and cytidine glycol, 5-hydroxyuridine, 3-hydroxymethyluridine, 3-methyluridine, O²-methyluridine, O²-ethyluridine, 5-aminouridine, O⁴-methyluridine, O⁴-ethyluridine, O⁴-isobutyluridine, O⁴-alkyluridine, 5-nitrosouridine, 5-(hydroxyalkyl)-uridine, and 5-(thioalkyl)-uridine, 1,N⁶-ethenoadenosine, 3-methyladenosine, and N⁶-methyladenosine, 8-hydroxyguanosine, O⁶-methylguanosine, O⁶-ethylguanosine, O⁶-isopropylguanosine, 3,N²-ethenoguanosine, O⁶-alkylguanosine, 8-oxo-guanosine, 2,N³-ethenoguanosine, and 8-aminoguanosineas well as derivatives/analogues thereof. Examples of suitable nucleoside precursors, and synthesis thereof, are described in further detail in USSN 20030119764. Generally, these agents are added to the replication or transcription reaction thereby mutating the sequence. Intercalating agents such as proflavine, acriflavine, quinacrine and the like can also be used.

Random mutagenesis of the polynucleotide sequence can also be achieved by irradiation with X-rays or ultraviolet light.

Methods for Selection of Nucleic Acids or Proteins/Peptides with an Altered Phenotype

The terms “altered phenotype”, “desired activity” and “altered activity” are generally used interchangeably herein.

In particular embodiments of the present invention, the mutated nucleic acid, or protein encoded thereby, is subjected to an assay for identifying an altered phenotype. Suitable procedures for identifying altered phenotypes include, but are not limited to, those described below.

Selection of RNA Molecules for Enhanced Expression

It will be appreciated by those skilled in the art that there are a number of different ways in which mutation(s) in RNA molecules can result in increased expression of the encoded protein. For example, the mutation(s) may lead to increased stability, preferred codon usage for the expression host, more effective protein synthesis due to increased access of the RNA to the translation machinery, or a combination of these factors.

In one embodiment, the selection procedure involves expressing the encoded protein in a cell or a cell-free translation system and panning against a molecule that binds to the encoded protein. In this embodiment, the procedure involves use of an appropriate concentration of the binding partner during the panning stage that allows any variant that can fold correctly and bind to be selected. To select for variants that have improved/altered RNA properties, careful selection of the concentration of the binding partner is important. In one embodiment, the translated proteins are mixed with defined amounts of soluble biotinylated binding partner such that the binding partner is in excess over the proteins but with the amount of the binding partner being at the concentration that is equivalent to the dissociation constant (Kd) of the wild-type encoded protein. The proteins that bind to the binding partner may then be selected using streptavidin-coated magnetic beads. Variants selected using the above panning strategy may then be subjected to a binding assay. The binding assay, for example an ELISA assay, is used to identify clones that give a response that is greater than the wild-type response. A very small proportion of the variants identified through this binding assay will exhibit an increased response because of an altered binding affinity. A larger proportion of the variants identified through this binding assay, however, will exhibit an increased response due to increased RNA stability or efficiency, while the binding affinity remains the same or similar as that of the protein derived from the wild type RNA molecule.

Selection of RNA Molecules for Enhanced Stability

Suitable processes for selection RNA molecules with increased stability will be known to those skilled in the art. For example, the stability may be assessed by the following procedures.

Measurement of RNA half life in vivo can be performed by growing host cells which produce the mutant RNA and extracting RNA from the host cells at various times throughout a given period. The level of the mutated RNA in the extracted sample can then be determined by Northern blot analysis (as described in Hambraeus et al, 2002) or by RT-PCR followed by Northern analysis.

Measurement of RNA levels in vitro can be performed by incubating RNA samples at room temperature for a given period of time. RNA levels can then determined both by reverse transcription-PCR(RT-PCR) using, for example, the SuperScript One-Step RT-PCR (Gibco-BRL) and by Southern analysis

In one embodiment the incubation may be performed in the presence of blood components or eukaryotic or prokaryotic extracellular lysates (such as those used to perform in vitro translations).

In another embodiment, incubation of the RNA may be conducted at elevated temperatures or in the present of ribonucleases.

The analysis of mRNA stability requires sensitive, precise, and reproducible measurement of specific mRNA sequences. Traditional techniques that can be used to quantify mRNA include methods based upon hybridization such as Northern blotting, solution hybridization, and RNase protection assays (Emory and Belasco, 1990). Amplification of individual RNA molecules by combining reverse transcription and the polymerase chain reaction (RT-PCR) can also be used and has been shown to be more sensitive because it exponentially amplifies small amounts of nucleic acid. This sensitivity enables the detection of mRNAs from small RNA samples (Schmittgen et al, 2000).

Recent advances in quantitative RT-PCR technology include the development of real-time quantitative PCR (Heid et al, 1996). Real-time PCR incorporates specific technology to detect the PCR product following each cycle of the reaction. Several methods are available to detect the DNA generated by real-time PCR including dual-la-beled fluorogenic hybridization probes (TaqMan probes) (Heid et al., 1996) and the SYBR green 1 minor groove DNA-binding dye (Wittwer et al, 1997). Real-time PCR allows sensitive detection of the DNA product, ensures detection during the linear range of amplification, eliminates the need for post-PCR analysis, and incorporates specialized software to simplify data analysis.

RNA Secondary Structure Analysis

RNA secondary structure can be analyzed using an RNA folding program. An example of such a program is available from the Microbiology website of the University of Adelaide, Adelaide, Australia (http://www.microbiology.adelaide.edu.au).

Protein/Peptide Display

One method of identifying proteins encoded by the mutant nucleic acids produced using the methods of the invention that possess a desired activity, such as binding to a predetermined biological macromolecule (e.g., a receptor), involves the screening of a large library of proteins/peptides for individual library members which possess the desired structure or functional property conferred by the amino acid sequence of the protein/peptide.

In addition to direct chemical synthesis methods for generating peptide libraries, several recombinant DNA methods also have been reported. One type involves the display of a peptide sequence, antibody, or other protein on the surface of a bacteriophage particle or cell (for review see Wittrup, 2001). Generally, in these methods each bacteriophage particle or cell serves as an individual library member displaying a single species of displayed peptide in addition to the natural bacteriophage or cell protein sequences. Each bacteriophage or cell contains the nucleotide sequence information encoding the particular displayed peptide sequence; thus, the displayed peptide sequence can be ascertained by nucleotide sequence determination of an isolated library member.

A well-known peptide display method involves the presentation of a peptide sequence on the surface of a filamentous bacteriophage, typically as a fusion with a bacteriophage coat protein. The bacteriophage library can be incubated with an immobilized, predetermined macromolecule or small molecule (e.g., a receptor) so that bacteriophage particles which present a peptide sequence that binds to the immobilized macromolecule can be differentially partitioned from those that do not present peptide sequences that bind to the predetermined macromolecule. The bacteriophage particles (i.e., library members) which are bound to the immobilized macromolecule are then recovered and replicated to amplify the selected bacteriophage subpopulation for a subsequent round of affinity enrichment and phage replication. After several rounds of affinity enrichment and phage replication, the bacteriophage library members that are thus selected are isolated and the nucleotide sequence encoding the displayed peptide sequence is determined, thereby identifying the sequence(s) of peptides that bind to the predetermined macromolecule (e.g., receptor). Such methods are further described in WO 91/17271, WO 91/18980, WO 91/19818 and WO 93/08278.

WO 93/08278 describes a recombinant DNA method for the display of peptide ligands that involves the production of a library of fusion proteins with each fusion protein composed of a first polypeptide portion, typically comprising a variable sequence, that is available for potential binding to a predetermined macromolecule, and a second polypeptide portion that binds to DNA, such as the DNA vector encoding the individual fusion protein. When transformed host cells are cultured under conditions that allow for expression of the fusion protein, the fusion protein binds to the DNA vector encoding it. Upon lysis of the host cell, the fusion protein/vector DNA complexes can be screened against a predetermined macromolecule in much the same way as bacteriophage particles are screened in the phage-based display system, with the replication and sequencing of the DNA vectors in the selected fusion protein/vector DNA complexes serving as the basis for identification of the selected library peptide sequence(s).

The displayed protein/peptide sequences can be of varying lengths, typically from 3-5000 amino acids long or longer, frequently from 5-100 amino acids long, and often from about 8-15 amino acids long. A library can comprise library members having varying lengths of displayed peptide sequence, or may comprise library members having a fixed length of displayed peptide sequence. Portions or all of the displayed peptide sequence(s) can be random, pseudorandom, defined set kernal, fixed, or the like. The display methods include methods for in vitro and in vivo display of single-chain antibodies, such as nascent scFv on polysomes or scFv displayed on phage, which enable large-scale screening of scFv libraries having broad diversity of variable region sequences and binding specificities.

A method of affinity enrichment allows a very large library of peptides and single-chain antibodies to be screened and the polynucleotide sequence encoding the desired peptide(s) or single-chain antibodies to be selected. The pool of polynucleotides can then be isolated and shuffled to recombine combinatorially the amino acid sequence of the selected peptide(s) (or predetermined portions thereof) or single-chain antibodies (or just V_{H}, V_{L}, or CDR portions thereof). Using these methods, one can identify a peptide or single-chain antibody as having a desired binding affinity for a molecule and can exploit the process of the invention to converge rapidly to a desired high-affinity peptide or scFv. The peptide or antibody can then be synthesized in bulk by conventional means for any suitable use (e.g., as a therapeutic or diagnostic agent).

In one embodiment, proteins encoded by nucleic acids obtained using the methods of the invention are displayed on the surface of the viruses. Systems for phage display are well known in the art and commercially available (see reviews by Felici et al, 1995; and Hoogenboom, 2002). Examples of phage display systems include, but are not limited to, M13 (Lowman et al, 1991); T7 (Novagen, Inc.); T4 (Jiang et al, 1997); lambda (Stolz et al, 1998); tomato bushy stunt virus (Joelson et al, 1997); and retroviruses (Buchholz et al, 1998).

In another embodiment, the proteins encoded by nucleic acids obtained using the methods of the invention are displayed on the surface of yeast. Suitable yeast display systems are known in the art (Boder and Wittrup, 1997; Cho et al, 1998).

In a further embodiment, the proteins encoded by nucleic acids obtained using the methods of the invention are displayed on the surface of a bacteria. Suitable bacterial display systems are known in the art (Stahl and Uhlen, 1997; Chen and Georgiou, 2002; Jung et al, 1998).

Yeast Two Hybrid Screening and Related Techniques

Proteins/peptides encoded by nucleic acids obtained using the methods of the invention can be used in a number of yeast based methods to detect protein-protein interactions. One well known system is the yeast two-hybrid system (Fields and Song, 1989) which has been used to identify interacting proteins and to isolate the corresponding encoding genes. In this system, prototrophic selectable markers which allow positive growth selection are used as reporter genes to facilitate identification of protein-protein interactions. Related systems which may be employed include the yeast three-hybrid system (Licitra and Liu, 1996) and the yeast reverse two-hybrid system (Vidal et al, 1996). Such procedures are known to those skilled in the art.

Cell-Free Continuous In Vitro Evolution Mutagenesis

In another use of the present invention, the methods can be applied to a cell-free continuous in vitro evolution mutagenesis system. In one example of cell-free continuous in vitro evolution, a system similar to that described in WO 99/58661 is utilized.

Thus, a cell-free continuous in vitro evolution method of the present invention comprises exposing mutant RNA molecules, produced directly or indirectly by the action of a polymerase in the presence of ribavirin, or a derivative/analogue thereof, to a translation system under conditions which result in the production of a population of mutant proteins. These mutant proteins are linked to the RNA from which they were translated forming a population of mutant protein/RNA complexes. This population of mutant protein/RNA complexes is screened for a desired biological activity such as binding to a target molecule. A mutant protein/RNA complex with the desired activity can be isolated and the sequence of the protein encoded by the RNA characterized by standard techniques.

The translation system for cell-free continuous in vitro evolution can be any such system known in the art, including those derived from prokaryotes or eukaryotes. Examples include the use of a rabbit reticulocyte lysates (He and Taussig, 1997) or an E. coli S-30 transcription translation mix (Mattheakis et al, 1994; Zubay, 1973). For mRNA synthesis in eukaryotic cells the mRNA is preferably capped which is achieved by adding an excess of diguanosine triphosphate; however, the rabbit reticulocyte system from the commercial suppliers Promega and Novagen have components in the system to make the addition of capping compounds unnecessary. The coupled transcription/translation system may be extracted from the E. coli mutator cells MUTD5-FIT (Irving et al, 1996) which bear a mutated DNAQ gene and therefore allow further random mutations introduced into DNA during replication as a result of proofreading errors. Addition of glutathione to the coupled system enhances correct folding of displayed proteins and therefore enhances subsequent binding and selection to counter-receptors or antigens.

Translation of the mutated mRNAs produces a library of protein molecules, preferably attached to the ribosome in a ternary ribosome complex which includes the encoding specific mRNA for the de novo synthesised protein (Mattheakis et al, 1994). Several methods are known to prevent dissociation of the mRNA from the translated protein and ribosome. For example, sparsomycin or similar compounds may be added; sparsomycin inhibits peptidyl transferase in all organisms studied and may act by formation of an inert complex with the ribosome (Ghee et al, 1996). Maintaining high concentrations of magnesium salts and lowering GTP levels may also contribute to maintaining the ribosome/mRNA/protein complex; in conjunction with the structure of the expression unit detailed above. A preferred means to maintain the ternary ribosome complex is the omission of the translation stop codon at the end of the coding sequence.

In addition, there are preferred requirements for the correct folding of the molecules in cell-free in vitro evolution systems. For prokaryotes protein disulphide isomerase (PDI) and chaperones may be used as well as a C-terminal anchor domain to ensure the correct folding. The latter is required as prokaryotic proteins are released from the ribosomes prior to folding (Ryabova et al, 1997) and therefore in situations in which the peptide is anchored to the ribosome the entire protein needs to be spaced from the ribosome. In contrast to this, in eukaryotic systems the protein is folded as it is synthesised and has no requirement for the prokaryote PDI and chaperones to be added. However, it has been found that addition of a specific range of glutathione concentrations is beneficial to the library selection by the enhanced display of correctly folded proteins on the ternary ribosome complexes.

Successive rounds of RNA replication produce libraries of RNA molecules which, upon translation, produce libraries of proteins. A target molecule-bound matrix (for example antigen-coated Dynabeads) may be added to the reaction to capture ternary ribosome complexes. The individual members in the library compete for the antigen immobilised on the matrix (Dynabeads). Molecules with a higher affinity will displace lower affinity molecules. At the completion of the process the complexes (mRNA/ribosomes/protein) attached to matrix (Dynabeads) may be recovered, cDNA may be synthesised from the mRNA in the complex and cloned into a vector suitable for high-level expression from the encoded gene sequence.

A recycling flow system (Spirin et al, 1988) may be applied to cell-free continuous in vitro evolution systems using a thermostated chamber to ensure supply of substrates (including ribosomes) and reagents and removal of non-essential products. All processes of cell-free continuous in vitro evolution may take place within this chamber including: coupled transcription and translation, mutating replication, display of the de novo synthesised protein on the surface of the ternary ribosome complex and competitive binding of the displayed proteins on the ternary ribosome complex to antigen to select those with the highest affinity binding. The unbound reagents, products and displayed proteins are removed by flushing with washing buffer and the bound ternary ribosome complexes are dissociated by increasing the temperature and omitting the magnesium from the buffer. This is followed with the addition of all the reagents necessary to carry out all the above steps except the washing buffer steps. Methods are available to prevent dissociation of the mRNA from the protein and ribosome such as the addition of sparsomycin or similar compounds, maintaining specific concentrations of magnesium salts and lowering GTP levels may also contribute to maintaining the ribosome/mRNA/protein complex as well as reducing the reaction temperature or omitting translational stop codons. By using vessels whose temperatures are controlled combined with a continuous flow capability, mRNAs from selected ribosomes may be dissociated from the ribosomes and further replicated, mutated and translated as the concentration of reagents important for the maintenance of the ribosome/mRNA/protein complex such as sparsomycin, Mg etc are varied.

The invention is hereinafter described by way of the following non-limiting examples.

### EXAMPLES

### Example 1

### Method for Preparing Q θ Replicase

Cloning and Expression of the Qbeta Replicase Viral Subunit

The oligonucleotides used as primers to amplify the Qθ replicase encoded sites for restriction enzyme digestion by the enzymes EcoRI and Not I and the sequences are shown here:

(SEQ ID NO: 2)

5′ TTACTCGCGGCCCAGCCGGCCATGGCCATGTCTAAGACAGCATCTT
CG

(SEQ ID NO: 3)

5′ TTTATAATCTGCGGCCGCCGCCTCGTGTAGAGACGCAAC

The PCR products were purified using QIAquick PCR Purification Kit (QIAGEN). The purified DNA was cloned into the EcoRI and NotI sites of the vector pGC using standard molecular biology techniques. The vector pGC and expression of recombinant therefrom has been described in the literature and is incorporated herein by reference. The process of the PCR amplification and cloning of the Qθ replicase gene into vectors and transformation into E. coli for expression of the enzyme will be obvious to those skilled in the art as will be the expression of the Qθ replicase gene in pGC which was induced by adding 1 mM ispropylthiogalatoside (IPTG) to the culture medium.

Expression and purification of the Qθ Replicase gene in the pBR322 based vector with the promoter ΣP_{L} was performed as detailed below. The rep14 Billeter strain was supplied by Christof Biebricher, Max Planck, Gottingen. The E. coli strain was grown in a 20 l fermentor in 2% nutrient broth, 1.5% yeast extract, 0.5% NaCl, 0.4% glycerol, 100 mg/l ampicillin with good aeration at 30° C. to an optical density of 2 (660 nM). After raising the temperature to 37° C., aeration was continued for 5 h. The cells were chilled on ice and harvested by centrifugation (yielding about 180 g wet cell mass).

Purification of Qβ Replicase

Buffer A: 0.05M Tris.HCl-buffer (pH 7.8), 1 mM β-mercaptoethanol, 20% v/v glycerol.

Buffer B: 0.05M HEPES.Na-buffer (pH 7.0), 1 mM β-mercaptoethanol, 20% v/v glycerol.

180 g harvested E. coli were homogenized with 360 ml 0.05M Tris.HCl buffer (pH7.8) 1 mM β-mercaptoethanol using an Ultra-Turrax T25 homogenizer (Janke and Kunkel; IKA Labortechnik). Lysozyme and EDTA were added to final concentrations of 100 μg/ml and 0.5 mM, respectively, and the solution was gently stirred at 0° C. for 30 min. 43.2 ml 8% Na deoxycholate, 0.86 ml phenylmethanesulfonyl fluoride (20 mg/ml in propanol-2), 0.54 ml Bacitracine (10 mg/ml), 0.54 ml 0.1M benzamidine, 11.9 ml 10% Triton-X-100 and 10 mM final concentration of MgCl₂, were added. The high viscosity was reduced by homogenizing (as above). Solid NaCl was added to a final concentration of 0.5M and 17.3 ml 0.3% polyethyleneimine (pH 8) was slowly stirred in for 20 min at 0° C. The suspension was centrifuged for 30 min at 15 000×g JA-17 or JA-10 rotor (Beckman J2-21 M/E). Following dilution of the supernatant with 5 volumes 0.05M Tris.HCl buffer (pH7.8), 1 mM β-mercaptoethanol, 360 ml DEAE cellulose slurry (Whatman DE52, equilibrated with buffer A) was added and slowly stirred at 0° C. for 20 min. This mixture was then left to sit for 40 min without stirring, and the supernatant was discarded by decanting. The sediment was suspended in buffer A, poured into a glass column of 2.5 cm diameter, washed with 1.41 0.05M Tris.HCl buffer (pH7.8); 1 mM β-mercaptoethanol, and eluted with 0.91 buffer A+180 mM NaCl. Fractions were collected and assayed for the presence of Qβ replicase using the following activity assay.

Activity Assay for Qβ Replicase:

This is a radioactive assay using the ¹⁴C-ATP and ssRNA template containing DHFR mRNA imbedded into the RQ-EGX recognition sequence. Scintillation counting was used to detect ¹⁴C incorporation into amplified RNA products. The standard reaction contained the following:

| | | |
|---|---|---|
| ssRNA template* | 50 | ng |
| rGTP | 10 | mM |
| rCTP | 10 | mM |
| rUTP | 10 | mM |
| rATP | 1 | mM |
| ¹⁴C-ATP | 0.05 | uCi |
| Tris-HCl (pH7.9) | 40 | mM |
| MgSO₄ | 18 | mM |
| Spermidine | 2 | mM |
| Dithiothreitol | 10 | mM |
| Rifampicin | 0.05 | ug |
| Qbeta Replicase | 100 | nM |

*the ssRNA template was generated with T7 RNA polymerase transcription from the SmaI linearized vector pEGX200

1-5 ul of each reaction was spotted onto GFC filter paper (Whatman; cut to 0.5×0.5 cm) and dried at room temperature. Filters were initially washed in 10 ml of ice-cold 10% TCA, with occasional stirring, for 15 min, followed by a second wash in 10 ml of ice-cold 5% TCA for 10 min. Finally, the filters were washed in ice-cold 70% ethanol for 30 min on ice, followed by a further wash in 100% ethanol for 10 min. Filters were dried at room temperature, transferred to 24 well scintillation plates (Optiplates; PACKARD) and 5 ml of scintillation fluid (Microscint™-40; PACKARD) was added prior to measuring count readings with a TopCount Microplate Scintillation Counter (PACKARD).

Further Purification of Qβ Replicase

The active fractions were pooled, diluted with one volume buffer A and applied to a 125 ml column of DEAE-Sepharose FF, equilibrated with buffer A+0.1M NaCl. The enzyme was eluted with a linear gradient (2×250 ml) of 0.1-0.4 M NaCl in buffer A. Active fractions were pooled and 39 g/10 ml of solid (NH₄)₂SO₄ was added to precipitate the enzyme. The pellet was collected by centrifugation and dissolved in 20 ml of Buffer B.

The enzyme was diluted until the conductivity was less than buffer B+0.2M NaCl and applied to a 10 ml Fractogel EMD SO₃⁻ column equilibrated with buffer B, and eluted with a linear gradient (2×50 ml) of 0.2-0.8M NaCl in buffer B. The active peaks, eluting at approximately 0.65M NaCl, were pooled, and the enzyme was precipitated with solid (NH₄)₂SO₄ (39 g/100 ml solution). The pellet was collected by centrifugation, dissolved in 1 ml buffer A+50% glycerol and stored at −80° C.

Following steps were performed at small scale according to Sumper and Luce (1975).

4 mg Qβ replicase was applied to a 1.6×14.5 cm column of QAE-Sephadex A-25 equilibrated with buffer A (diluted or dialysed to remove salt), and eluted with a (2×200 ml) gradient of 0.05-0.25M NaCl in buffer A. The two separated peaks of core and holo enzyme were pooled, diluted 1:1 with buffer A and applied to QAE-Sephadex columns, 2 ml for core, 6 ml for holo replicase, respectively. The columns were washed with buffer A+50% glycerol, and the replicase was eluted with buffer A+50% glycerol+0.2 M (NH₄)₂SO₄. The active fractions were stored at −80° C. Extreme care was taken to avoid contamination of the equipment with RNA.

### Example 2

### Method for Performing Replication and Mutagenesis of RNA by Qbeta Replicase

Qβ-replicase amplification of RNA templates is used to both amplify and to introduce mutations into the RNA.

The method was as follows:

| | | |
|---|---|---|
| ssRNA template | 20-100 | ng* |
| rGTP | 10-25 | mM* |
| rCTP | 10-25 | mM* |
| rATP | 10-25 | mM* |
| rUTP | 10-25 | mM* |
| Tris-HCl (pH6-9*) | 40 | mM |
| MgSO₄ | 6-21 | mM* |
| Spermidine | 2 | mM |
| Dithiothreitol | 10 | mM |
| Qbeta Replicase | 100 | nM |

The reaction is incubated at 25-55° C.* for 0.5-24 hrs*.
*concentrations and conditions vary depending on the gene sequence being amplified and the level of mutagenesis required.

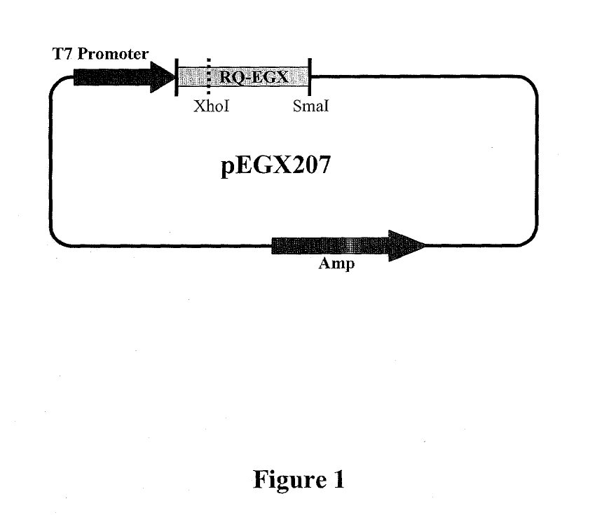

The RNA template may be produced using a suitable vector such as pEGX207 (FIG. 1).

### Example 3

### Production and Use of Alternative RNA Replicases

Phi6 RNA Replicase (P2) amplification of RNA templates is used to amplify and to introduce mutations into the RNA.

The method was as follows:

| ssRNA template | 20-100 | ng* |
|---|---|---|
| rGTP | 1-10 | mM* |
| rCTP | 1-10 | mM* |
| rATP | 1-10 | mM* |
| rUTP | 1-10 | mM* |
| Tris-HCl (pH6-9.5*) | 50 | mM |
| NH4OAc | 80 | mM |
| PEG4000 | 6% | (w/v) |
| MgSO4 | 1-10 | mM* |
| Triton X-100 | 0.1% | |
| EDTA | 0.1 | mM |
| MnCl2 | 0.1-5 | mM* |
| Dithiothreitol | 2 | mM |
| Phi6 Replicase (P2) | 100 | nM |

The reaction is incubated at 25-37° C.* for 0.5-24 hrs*.
*concentrations and conditions vary depending on the gene sequence being amplified and the level of mutagenesis required.

### Example 4

### Method for Preparing Phi6 P2 Replicase

Cloning and Expression of the Phi6 Viral Replicase (P2)

Overlapping oligonucleotides were used to construct the P2 replicase sequence using methodology that will be obvious to those skilled in the art. The gene sequence was purified using QIAquick PCR Purification Kit (QIAGEN). The purified DNA was cloned into the EcoRI and NotI sites of the vector pGC using standard molecular biology techniques. The vector pGC and expression of recombinant therefrom has been described in the literature and is incorporated herein by reference. The process of the PCR amplification and cloning of the Qθ replicase gene into vectors and transformation into E. coli for expression of the enzyme will be obvious to those skilled in the art as will be the expression of the P2 replicase gene in pGC which was induced by adding 1 mM ispropylthiogalatoside (IPTG) to the culture medium.

Expression and purification of the P2 Replicase gene in the pBR322 based vector with the promoter ΣP_{L} was performed as detailed below. The E. coli strain BL21(DE3) was supplied by Novagen. The cells were grown in a 20 l fermentor in 2% nutrient broth, 1.5% yeast extract, 0.5% NaCl, 0.4% glycerol, 100 mg/l ampicillin with good aeration at 30° C. to an optical density of 2 (660 nM). After raising the temperature to 37° C., aeration was continued for 5 h. The cells were chilled on ice and harvested by centrifugation (yielding about 180 g wet cell mass).

Purification of Phi6 Replicase

500 g harvested E. coli were homogenized with 1 litre of 0.05M Tris.HCl buffer (pH8.7) 1 mM mercaptoethanol in a high-speed blender. Lysozyme and EDTA are added to final concentrations of 100 Tg/ml and 0.5 mM, respectively, and the solution was gently stirred at 0° C. for 30 min. 120 ml 8% Na deoxycholate, 2.4 ml phenylmethanesulfonyl fluoride (20 mg/ml in propanol-2), 1.5 ml Bacitracine (10 mg/ml), 1.5 ml 0.1M benzamidine, 33 ml 10% Triton-X-100 were added and the solution adjusted with MgCl₂ to 10 mM final concentration. The high viscosity was reduced by blending at high speed. Solid NaCl was added to a final concentration of 0.5M and 48 ml 0.3% polyethyleneimine (pH 8) was stirred in. After stirring for 20 min at 0° C. the suspension was centrifuged for 2.5 h at 12,000×g (Beckman J2-21 M/E).

The following steps were performed at small scale according to Makeyev and Bamford (2000, The EMBO J 19, 124-1133).

The supernatant fraction was loaded onto a Cibacron Blue 3GA dye affinity column (Sigma). Proteins bound to the column were eluted with 500 mM NaCl, 50 mM Tris-HCl pH 8.0 and 1 mM EDTA. Fractions containing P2 were pooled and diluted 5-fold with ice-cold distilled water and applied onto a heparin agarose column (Sigma). Proteins were eluted with a linear 0.1-1 M NaCl gradient buffered with 50 mM Tris-HCl pH 8.0 and 1 mM EDTA. Fractions containing P2 were pooled and diluted 10-fold with 20 mM Tris-HCl pH 8.0, filtered and passed through a Resource Q column at 20° C. (Pharmacia). Elution of the bound proteins was performed with a 0-0.5 M NaCl gradient buffered with 50 mM Tris-HCl pH 8.0 and 0.1 mM EDTA. Purified P2 protein was stored in buffer A+50% glycerol. The solution was stored at −80° C.

### Example 5

### Generation of Mutant RNA Molecules with Increased Stability

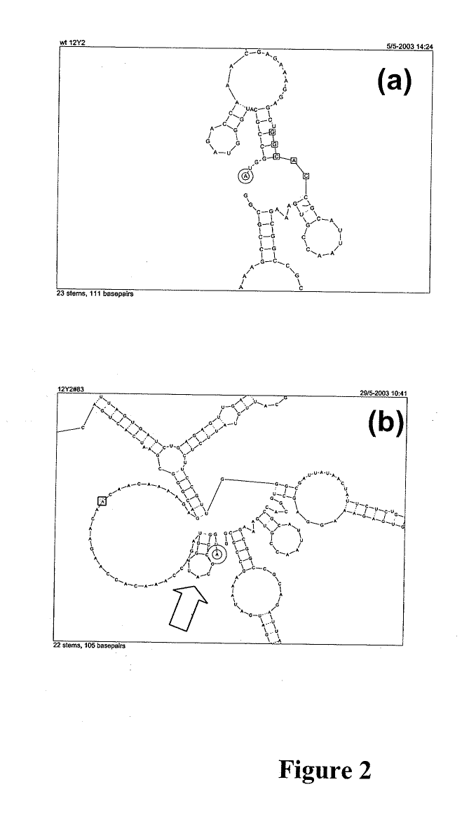

The present inventors compared the nucleotide sequences of a starting RNA encoding a wild type binding protein (12Y-2) and a mutant sequence found to express the encoded protein at a higher level, as shown in Example 6. This mutant sequence contained no mutations that altered the amino acid sequence of the encoded protein, leading to the conclusion that increased protein expression observed was caused by increase in RNA stability, an increase in ease of translation of the RNA, or some combination of these. The present inventors have used a computer program (RNAdraw v1.1) to compare the potential RNA structure of these two RNAs. The predicted structures are shown in FIG. 2. This analysis surprisingly showed that the introduction of the observed nucleotide change allowed the formation of a small stem loop structure (marked with an arrow) close to the initiating AUG codon (FIG. 2b). The structure is not present in the wild type RNA (FIG. 2a). The mutant RNA had a mutation located approximately 30 nucleotides away from the start. This is consistent with computational modelling of mRNA turnover by Cao and Parker (2001) that indicated that the 3′-to-5′ degradation of mRNA correlates to mRNA-specific rates of degradation that are dependent on the 5′ structure of the mRNA. At the same time, the altered environment of the AUG codon may affect its access to the translation machinery of the ribosome.

### Example 6

### Protocol for Ribosome Display and RNA Stability/Efficiency Selection

The example provided below utilizes the apical membrane antigen 1 (AMA-1) which is a single transmembrane domain protein that is essential for binding and penetration of the malaria (Plasmodium falciparum) parasite (merozoite) into red blood cells. Antibodies to AMA-1 block merozoite invasion. The single domain antibody (NAR) designated 12Y-2 binds to AMA-1 and prevents merozoite invasion.

Buffers Used In Ribosome Display

Buffer A; Phosphate Buffered Saline (pH 7.4); 50 mM MgCl₂

Buffer B: Buffer A; 0.05% (v/v) Tween 20

Buffer C: Buffer B; 2.5 mg/ml heparin

Buffer E: Buffer A; 10% (w/v) Skim milk powder

In Vitro Translation Reaction

All steps in the protocol used ice-cold solutions and were performed on ice where possible.

Translation Mix

20 units RNasin

100 mM KCl*

2 mM Mg Acetate*

50 μM of each amino acid,

33 μl of Flexi rabbit reticulocyte lysate (Promega),

1-10 μg 12Y-2 RNA#

dH₂O up to a final translation mix volume of 50 μl.

*Concentrations were determined empirically as Mg2+ and K+ concentrations effect the efficiency of the in vitro translation with the efficiency of the ribosome display directly related to the amount of protein produced in the translation mix.

#12Y-2 RNA was generated using Qθ replicase mutagenesis.

The translation mix was incubated at 30° for 30 min and then diluted with 200 ul of ice-cold Buffer C and 64 ul ice-cold Buffer E. 100 ul aliquots were placed into panning tubes containing 50-300 nM biotinylated AMA-1 (the binding constant of 12Y-2 to AMA-1 is estimated at 250+/−100 nM so a range of concentrations of biotinylated AMA-1 was used to ensure that the correct concentration was used) and incubated on ice for 60 min to allow correctly folded 12Y-2/ribosome/RNA complexes to bind to biotinylated AMA-1.

12Y-2/ribosome/RNA complexes bound to biotinylated AMA-1 were recovered using streptavidin-coated magnetic beads, washed twice with Buffer B and twice in Buffer A. Beads (with the associated AMA-1/12Y-2/ribosome/RNA complexes) were used directly in a one step RT-PCR reaction (Invitrogen) using a primer pair specific for the 12Y-2 sequence. Amplified cDNA was concurrently digested with NcoI and NotI, ligated into pGC4C26H and transformed into E. coli (strain HB2151).

Individual clones are grown in 200 ul nutrient broth cultures containing 100 ug/ml ampicillin for 6 hrs at 30° C. and then induced for protein synthesis by the addition of 1 mM IPTG and incubated at 20° C. overnight. The supernatant from each individual clone was transferred to a 96 well tray that had been coated with AMA-1. Bound 12Y-2 was visualized with an anti-Flag antibody conjugated to horse radish peroxidase (Sigma). The response of clones were compared to the response of wild-type 12Y-2. Clones with an increased response (2-fold over that of the wild-type) were selected and analyzed for increased expression. Selected clones were grown in 80 ml nutrient broth containing 100 ug/ml ampicillin to an OD600 reading of 1.0 before the addition of 1 mM IPTG. 1 ml samples were removed at 0, 2, 4, 7 and 16 hrs following the addition of IPTG. The samples were centrifuged to remove the bacterial cells. 10 ul of the culture supernatant was run on a SDS polyacrylamide gel, transferred to a nylon membrane and probed with an anti-flag antibody conjugated to horse radish peroxidase (Sigma).

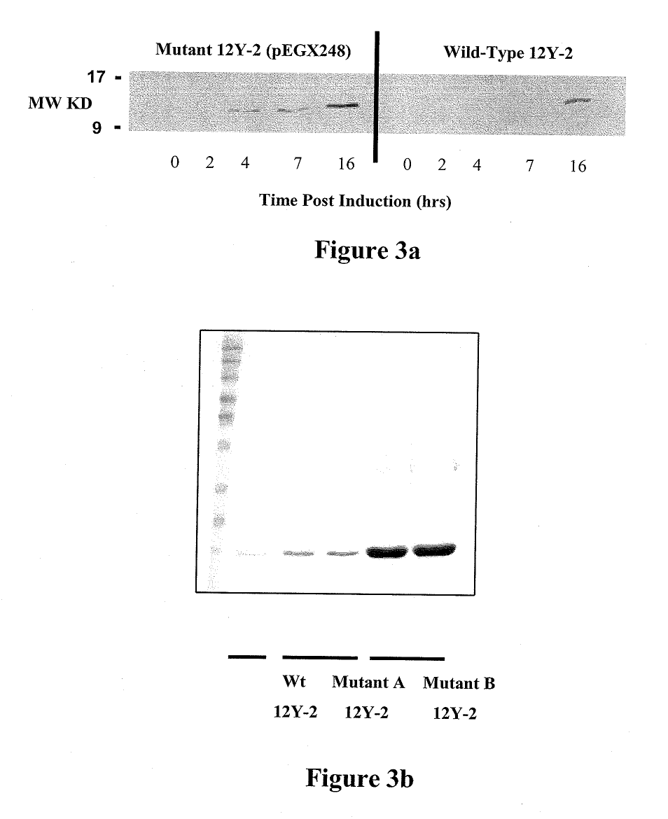

The data in FIG. 3a illustrates that mutant protein is expressed to detectable levels within 4 hours post-induction while protein from the unmutated gene can not be detected until 16 hours post-induction. The RNA species giving rise to the proteins exemplified in this example both code for the same amino acid sequence and both proteins preserve binding to AMA-1. However, the nucleotide sequence in the mutant clone is altered to include silent mutations. In a second example, the protein products of two different mutant RNAs derived from the wild type sequence encoding 12Y-2 are seen to be expressed, as demonstrated after purification, at higher levels compared to protein encoded by the wild-type RNA sequence (FIG. 3b). The data indicates that the process is introducing and selecting for mutations which stabilize the RNA and/or allow the RNA to be more easily expressed, resulting in higher levels of protein production.

It is well known that production of foreign proteins in a variety of expression systems is often limited by the rarity of certain tRNAs that are abundant in the organisms from which the foreign protein originated. High-level expression of foreign proteins in a variety of systems including E Coli can deplete the pool of rarer tRNAs and lead to a stalling of translation which leads to a reduction in the amount of product being produced. By altering the codon bias of the starting RNA to use more abundant tRNAs without altering the amino acid sequence will lead to improved high-level expression of foreign proteins in a variety of expression systems as indicated in the present example.

### Example 7

### Method for Real Time RT-PCR

The procedure outlined below can be used to measure RNA stability. More specifically, upon producing mutant RNA molecules using the methods of the invention, the resulting products can be exposed to conditions which promote RNA degradation, and then the presence of remaining RNA molecules determined using, for example, the following RT-PCR method. Mutant RNAs which result in higher levels of amplification product indicate which mutant RNA molecules are more stable than the wild-type molecule.

RNA Extraction and Reverse Transcription.

DNA-free (residual plasmid DNA was digested by incubating the RNA solution with 15 units of RNase-free DNase I (Promega) in 40 mM Tris.HCl (pH 8), 10 mM MgCl₂ and 1 mM CaCl₂ for 10 min at 37° C. followed by 15 min at 65° C. to inactivate the Dnase I), 12Y-2 RNA was isolated from solution (either from Flexi rabbit reticulocyte lysate, serum or buffers) with the RNeasy RNA isolation kit (Qiagen). The RNA solution was used in a reverse transcription reaction as follows: 0.1-1 ug RNA was used in a reaction containing 50 mM Tris-HCl (pH 8.3), 10 mM dithiothreitol, 10 pmole sequence specific primers, 3 mM MgCl₂, 0.5 mM deoxynucleotide triphosphates, 3 units of RNasin (Promega) and 50 units of RNase H minus Moloney murine leukemia virus reverse transcriptase (Promega). The reactions were incubated at 42° C. for 45 min followed by a 3-min incubation at 90° C. to denature RNA secondary structure. The cDNA was quantitated using the real-time PCR using TaqMan.

Real-Time PCR

Reactions for the real-time PCR using TaqMan detection (PE Biosystems) consisted of 1×TaqMan buffer A; 200 nM dATP, dGTP, and dCTP; 400 nM dUTP; 4.5 mM MgCl₂; 0.25 units of uracil N-glycosylase; 0.6 units of AmpliTaq Gold DNA polymerase; 250 nM forward and reverse primers: 250 nM dual-labeled fluorogenic hybridization probe: 5 ul of a 1:10 dilution of the cDNA.

Real-time PCR was performed in the PE Biosystems GeneAmp 5700 sequence detection system in a MicroAmp 96-well plate capped with Micro-Amp optical caps. The reactions were incubated at 50° C. for 2 min to activate the uracil N′-glycosylase and then for 10 min at 95° C. to inactivate the uracil N-glycosylase and activate the Amplitaq Gold polymerase followed by 40 cycles of 15 s at 95° C., 30 s at 55° C., and 30 s 72° C.

RNA degradation was determined by normalizing the amount of RNA from the degradation conditions to an identical concentration of RNA held in 10 mM Tris buffer pH7.5.

### Example 8

### Introduction of Mutations in the Dihydrofolate Reductase Gene (DHFR) Using a DNA Dependent RNA Polymerase in the Presence of Ribavirin

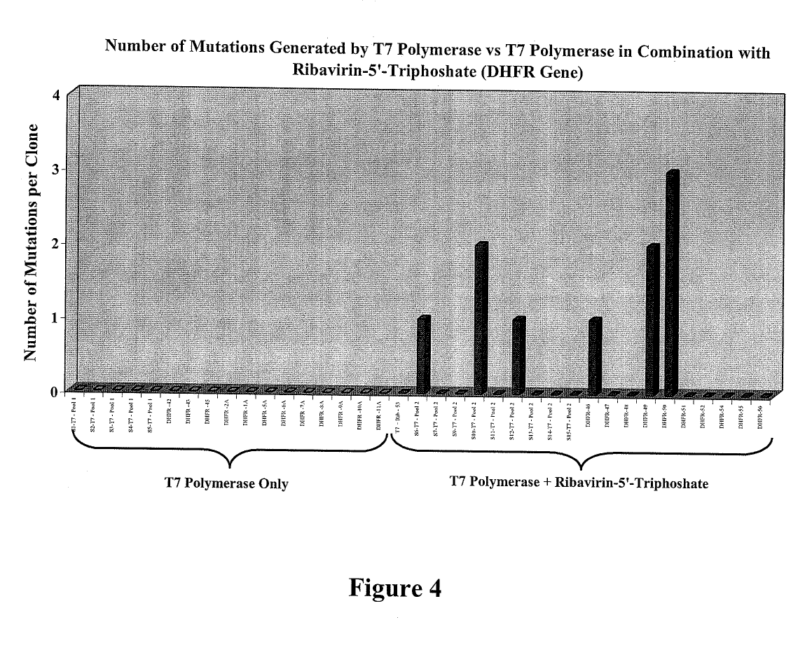

T7 RNA polymerase with the addition of 1000TM ribavirin 5′ triphosphate (dTRV) was used to transcribe dihydrofolate reductase gene (DHFR) (−) RNA from the DHFR gene on plasmid pEGX200. The RNA was subsequently converted to cDNA using M-MuLV-reverse transcriptase, cut with appropriate restriction enzymes, and inserted into pPCR-Script AMP(+) (Stratagene). The plasmid mixture was used to transform E. coli and clones containing DHFR cDNA were selected at random and the DHFR region sequenced. The sequence data indicates the number and relative gene position of each nucleotide mutation found (Table 2 and FIG. 4). Transcription of the DHFR sequence in the presence of ribavirin resulted in numerous mutations in the clones produced in these experiments. In contrast, no mutations were observed in control

TABLE 2
Clones containing DHFR cDNA were selected at random
and sequenced.

| | | | Relative |
|---|---|---|---|
| | | | Mutation |
| | | | Position |
| | | No of | DHFR |
| | | Point | Gene |
| Treatment | Clone No | Mutations | (1-472 bp) |
| T7 Polymerase | S1-T7-Pool 1 | 0 | |
| T7 Polymerase | S2-T7-Pool 1 | 0 | |
| T7 Polymerase | S3-T-Pool 1 | 0 | |
| T7 Polymerase | S4-T7-Pool 1 | 0 | |
| T7 Polymerase | S5-T-Pool 1 | 0 | |
| T7 Polymerase | DHFR-42 | 0 | |
| T7 Polymerase | DHFR-43 | 0 | |
| T7 Polymerase | DHFR-45 | 0 | |
| T7 Polymerase | DHFR-2A | 0 | |
| T7 Polymerase | DHFR-1A | 0 | |
| T7 Polymerase | DHFR-5A | 0 | |
| T7 Polymerase | DHFR-6A | 0 | |
| T7 Polymerase | DHFR-7A | 0 | |
| T7 Polymerase | DHFR-8A | 0 | |
| T7 Polymerase | DHFR-9A | 0 | |
| T7 Polymerase | DHFR-10A | 0 | |
| T7 Polymerase | DHFR-11A | 0 | |
| T7 Polymerase & Ribavirin* | T7 + Rib-53 | 0 | |
| T7 Polymerase & Ribavirin* | S6-T7-Pool 2 | 1 | 345 |
| T7 Polymerase & Ribavirin* | S7-T7-Pool 2 | 0 | |
| T7 Polymerase & Ribavirin* | S9-T7-Pool 2 | 0 | |
| T7 Polymerase & Ribavirin* | S10-T7-Pool 2 | 2 | 93, 302 |
| T7 Polymerase & Ribavirin* | S11-T7-Pool 2 | 0 | |
| T7 Polymerase & Ribavirin* | S12-T7-Pool 2 | 1 | 263 |
| T7 Polymerase & Ribavirin* | S13-T7-Pool 2 | 0 | |
| T7 Polymerase & Ribavirin* | S14-T7-Pool 2 | 0 | |
| T7 Polymerase & Ribavirin* | S15-T7-Pool 2 | 0 | |
| T7 Polymerase & Ribavirin* | DHFR-46 | 1 | 193 |
| T7 Polymerase & Ribavirin* | DHFR-47 | 0 | |
| T7 Polymerase & Ribavirin* | DHFR-48 | 0 | |
| T7 Polymerase & Ribavirin* | DHFR-49 | 2 | 112, 354 |
| T7 Polymerase & Ribavirin* | DHFR-50 | 3 | 136, 270, |
| | | | 280 |
| T7 Polymerase & Ribavirin* | DHFR-51 | 0 | |
| T7 Polymerase & Ribavirin* | DHFR-52 | 0 | |
| T7 Polymerase & Ribavirin* | DHFR-54 | 0 | |
| T7 Polymerase & Ribavirin* | DHFR-55 | 0 | |
| T7 Polymerase & Ribavirin* | DHFR-56 | 0 | |

*Ribavirin was in the form of ribavirin 5′-triphosphate

Reactions where the DHFR sequence was transcribed by T7 RNA polymerase in reactions lacking ribavirin.

The data indicates that dTRV causes a significant increase in the number of point mutations seen with T7 RNA polymerase. The T7 RNA polymerase error rate is about 0.01% (i.e. 1 base change in 10,000 nucleotides) and would not be expected to generate significant mutations within a sequence of the length of the DHFR gene (472 bp) as indicated by the data. With the addition of dTRV, 6 out of 20 clones had one or more point mutations (Table 2 and FIG. 4); a clear demonstration that T7 polymerase in combination with dTRV is introducing errors during transcription.

### Example 9

### Introduction of Mutations in the β-Lactamase Gene (Bla) Using a DNA Dependent RNA Polymerase in the Presence of Ribavirin

T7 RNA polymerase with the addition of 1000TM ribavirin 5′ triphosphate (dTRV) was used to transcribe (−) RNA from the bla gene θ-lactamase gene for antibiotic resistance) on plasmid pEGX205. The bla RNA was subsequently converted to cDNA using M-MuLV-reverse transcriptase, amplified with Pfu DNA polymerase, cut with appropriate restriction enzymes, and cloned into the plasmid pEGX212. Clones resistant to increased concentrations of cefotaxime were selected and sequenced (the minimum inhibitory concentration [MIC] for the wild-type bla gene was 0.02 ug/ml). With one round of the mutation/selection process, a mutant bla sequence (clone CefR E1) containing two amino-acid substitutions was selected that showed a 2000-fold increase in cefotaxime resistance over the wild-type (Table 3). When clone CefR E1 RNA was taken through a second round of T7 polymerase/dTRV mutagenesis, 3 clones containing 3 or more mutations were selected with one clone (F-E1-1) showing a 10,000-fold increase in cefotaxime resistance over the wild-type. No resistant mutants were isolated in the mutation/selection experiments that used T7 polymerase alone. The data indicates that T7polymerase/dTRV mutagenesis is an effective tool for generating diverse libraries of nucleic acids that can be used for the maturation (evolution) of proteins.

TABLE 3
Application of T7 polymerase/ribavirin-5′-triphosphate mutagenesis to
mature (increase) resistance to the antibiotic cefotaxime conferred
by the TEM-1 Θ-lactamase (bla) gene expressed in bacteria.

| | | | | |
|---|---|---|---|---|
| | No. | | | MIC^{#} |
| Clone | Muta- | Position & | | (ug/ |
| Code | tions | Change | AA Change | ml) |

Round 1 - Mutations

| | | | | |
|---|---|---|---|---|
| CefR E1 | 2 | G238S, E104K, | Gly->Ser, Glu->Lys | 40 |

Round 2 - Mutations (Starting Clone Used Was
CefR E1 From Round 1)

| | | | | |
|---|---|---|---|---|
| CefR | 4 | G238S*, E104K*, | Gly->Ser, Glu->Lys, | 100 |
| E1 (1) | | H153R, a2502g | His -> Arg, “SD”** | |
| E-E1 (1) | 3 | G238S*, E104K*, | Gly->Ser, Glu->Lys, | 80 |
| | | F24S | Phe -> Ser | |
| F-E1 (1) | 3 | G238S*, E104K*, | Gly->Ser, Glu->Lys, | 200 |
| | | M182T | Met -> Thr | |

^{#}Wild-type cefotaxime minimum inhibitory concentration (MIC) was 0.02 ug/ml
*Mutations carried over from round 1
**“SD” means a mutation in the Shine-Dalgarno sequence

### Example 10

### Testing of Further Polymerases for the Effect of Ribavirin on Mutation Frequencies

DNA Dependent RNA Polymerases

Three DNA dependent RNA polymerases were tested using a DNA template, namely T7 RNA polymerase, T3 RNA polymerase and SP6 RNA polymerase (Table 4). Linear DHFR (dihydrofolate reductase) DNA template (0.2 ng-1.0 ug) was combined with 1 mM each of rCTP, rGTP, rATP and rUTP, 40 mM Tris-HCl (pH 7.9 at 25° C.), 10 mM NaCl, 6 mM MgCl₂, 2 mM Spermidine, 3 U RNasin (Promega), 4 U/ml inorganic pyrophosphatase (NEB), 10 mM DTT (Promega), 1000-2000 uM+/−dTRV and 40 U of T7, T3 or SP6 RNA Polymerase (Promega) and incubated at 37° C. for 2-18 hrs.

The resultant RNA was DNase-treated as described in Example 7, cleaned using RNeasy Mini Kit (QIAGEN) and the RNA resuspended in RNase-free dH₂O.

The RNA solution was used in a one step reverse transcription reaction (SuperScript One Step RT-PCR-Invitrogen) by combining SuperScript III One Step Reaction Mix, 10 pmole DHFR specific forward and reverse primers, 5 mM Mg2⁺, 0.1-1.0 ug RNA, and SuperScript II reverse transcriptase/Platinum Taq mix. The reactions were incubated at 50° C. for 45 min then at 94° C. for 2 min to inactivate the reverse transcriptase followed by 40 cycles of a PCR amplification (94° C. for 30 sec/54° C. for 1 min/68° C. for 2 min) and 1 cycle of 68° C. for 7 min.

PCR products that corresponded to the DHFR gene product were gel purified using standard procedures (NucleoSpin gel elution kit: Macherey-Nagel). The PCR fragments were end polished by incubating at 72° C. for 30 min with the addition of 5 mM dATP, dGTP, dCTP, dTTP, polishing buffer (Stratagene) and 0.5 U cloned Pfu DNA polymerase (Stratagene). The polished PCR fragments were blunt end cloned into pPCR-Script AMP(+) (Stratagene). Clones containing DHFR DNA were selected at random and sequenced.

For each DNA-dependent RNA polymerase, an increased mutation rate was observed with ribavirin. Furthermore, increasing the ribavirin concentration from 1000 μM to 2000 μM resulted in an increase in the number of mutations when using the SP6 polymerase indicating a dose dependent effect. The SP6 polymerase was the only enzyme that was tested with two concentrations of ribavirin, however, it is expected that a similar dose dependent effect will also obtained using other polymerases. Thus, use of ribavirin, or derivatives/analogues thereof, at concentrations above 2000 μM can be expected to further increase mutation rates in systems where mutations rates are already above baseline and to induce mutations in systems previously refractory to mutation.

RNA Dependent DNA Polymerases

Three RNA dependent DNA polymerases were tested using an RNA template, namely SuperScript III (from invitrogen), AMV and M-MuLV (Table 4).

For AMV reverse transcriptase, DHFR RNA template (0.2 ng-2.0 ug) was combined with 10 pmole DHFR specific forward and reverse primers and incubated at 70° C. for 10 min, then placed on ice before adding 20 nMol each of dCTP, dGTP, dATP and dTTP, 50 mM Tris-HCl (pH 8.5 at 20° C.), 8 mM MgCl₂, 30 mM KCl, 1 mM DTT (Promega), 25 U RNasin RNase inhibitor (Promega), 1000 uM+/−dTRV and 40 U of AMV reverse transcriptase (Roche). The reaction mix was incubated at 42° C. for 60 min. The reaction was inactivated by heating to 70° C. for 10 min.

For M-MuLV reverse transcriptase, DHFR RNA template (0.2 ng-2.0 ug) was combined with 10 pmole DHFR specific forward and reverse primers and incubated at 70° C. for 10 min, then placed on ice before adding 20 nMol each of dCTP, dGTP, dATP and dTTP, 50 mM Tris-HCl (pH 8.3 at 37° C.), 6 mM MgCl₂, 40 mM KCl, 1 mM DTT (Promega), 25 U RNasin RNase inhibitor (Promega), 1000 uM+/−dTRV and 40 U of M-MuLV reverse transcriptase (Roche) and incubated at 37° C. for 60 min. The reaction was inactivated by heating to 70° C. for 10 min.

For Superscript III, DHFR RNA template (0.1 ng-5 ug) was combined with 2 pmole DHFR specific forward and reverse primers and 20 nMol each of dCTP, dGTP, dATP and dTTP. The mixture was heated to 65° C. for 5 min, then placed on ice before adding First Strand Buffer (Invitrogen), 1 mM DTT (Promega), 25 U RNasin RNase inhibitor (Promega), 1000 uM+/−dTRV and 200 U of SuperScript III Reverse Transcriptase (Invitrogen) and incubated at 50° C. for 60 min. The reaction was inactivated by heating to 70° C. for 10 min.

Products from the reverse transcription reactions were amplified using a high-fidelity DNA polymerase. The cDNA was mixed with 20 mM Tris-HCl (pH 8.8 at 25° C.), 10 mM KCl, 10 mM (NH₄)₂SO₄, 0.1% Triton X-100, 10 pmole DHFR specific forward and reverse primers and 4 U Deep Vent DNA polymerase (NEB). The reactions were incubated in a thermal cycler at 94° C. for followed by 40 cycles of a PCR amplification (94° C. for 30 sec/68° C. for 2 min) and 1 cycle of 68° C. for 7 min.

Mutation frequencies were significantly increased in the presence of ribavirin, when nucleic acid was copied using the AMV and M-MuLV polymerases. Whilst a significant level of mutations was not observed when ribavirin was combined with SuperScript III polymerase, one mutation was noted in the presence of ribavirin whereas no mutations were observed in the absence of ribavirin, confirming that ribavirin is able to act as a mutagen during copying of RNA into DNA.

Tth DNA Dependent DNA Polymerase

DHFR DNA template (0.1-50 ng) was amplified using Tth DNA polymerase (BioTech). The DNA was mixed with 20 mM Tris-HCl (pH 8.8 at 25° C.), 10 mM KCl, 10 mM (NH₄)₂SO₄, 0.1% Triton X-100, 10 pmole DHFR specific forward and reverse primers and 4 U Deep Vent DNA polymerase (NEB). The reactions were incubated in a thermal cycler at 94° C. for followed by 40 cycles of a PCR amplification (94° C. for 30 sec/68° C. for 2 min) and 1 cycle of 68° C. for 7 min.

The Tth DNA dependent DNA polymerase was tested using a DNA template where the mutation frequency was significantly increased in the presence of ribavirin (Table 4).

### Example 11

### Use of Qβ Replicase and Ribavirin for the Mutation and Selection of β-Lactamase Enzyme with Improved Resistance to Cefotaxime

Mutation and selection of β-lactamase with improved resistance to cefotaxime is carried out essentially as follows. A gene encoding bacterial β-lactamase is ligated into the RQ135 sequence contained on a DNA vector and transcribed using T7 RNA polymerase. The transcripts are then amplified using Qβ replicase in the presence of ribavirin under the conditions outlined below:

RNA template (10 ng)

ATP (200 μM)

CTP (200 μM)

GTP (200 μM)

UTP (200 μM)

Ribavirin (1000 TM)

Replicase buffer (40 mM Tris-HCl pH 7.9, 21 mM MgCl₂, 10 mM DTT, 2 mM spermidine)

Qβ replicase (1.50 pmol)

TABLE 4
Mutation frequencies produced by a variety of polymerases in the presence of ribavirin,
added to the reactions in the form of ribavirin triphosphate.

| | | | | | | | | FOLD INCREASE |
|---|---|---|---|---|---|---|---|---|
| | | | | RIBAVIRIN | | | | IN MUTATION |
| | NO RIBAVIRIN | | | Ribavirin | | | | FREQUENCY BY |
| | Bases | Mutations | Mutation | Concentration | Bases | Mutations | Mutation | RIBAVIRIN |
| ENZYME | Sequenced | Identified | Frequency | (μM) | Sequenced | Identified | Frequency | FREQUENCY |
| T3 DNA Dependent | 8640 | 3 | 1:2880 | 1000 | 8160 | 9 | 1:907 | 3.2x |
| RNA Polymerase | | | | | | | | |
| SP6 DNA Dependent | 16370 | 9 | 1:1813 | 1000 | 9120 | 11 | 1:829 | 2.2x |
| RNA Polymerase | | | | 2000 | 7200 | 12 | 1:600 | 3x |
| AMV RNA | 9600 | 3 | 1:3200 | 1000 | 9120 | 11 | 1:829 | 3.9x |
| Dependent DNA | | | | | | | | |
| Polymerase | | | | | | | | |
| M-MuLV RNA | 7680 | 0 | 0:7680 | 1000 | 8640 | 6 | 1:1440 | Greater than 6x |
| Dependent DNA | | | | | | | | |
| Polymerase | | | | | | | | |
| Super Script III RNA | 9600 | 0 | 0:9600 | 1000 | 9600 | 1 | 1:9600 | Greater than 1x |
| Dependent DNA | | | | | | | | |
| Polymerase | | | | | | | | |
| Tth DNA Dependent | 6720 | 5 | 1:1344 | 1000 | 6240 | 13 | 1:480 | 2.8x |
| DNA Polymerase | | | | | | | | |

Transcripts amplified by Qβ replicase and ribavirin, together with a control population of transcripts that are processed only with Qβ replicase, are then converted into DNA by RT-PCR. Primers for RT-PCR are:

(SEQ ID NO: 4)

5′ CGAGGCGGCCGCGGTCATGAGATTATCAAAAAGG
and

(SEQ ID NO: 5)

5′ TCGAGCCATGGCTCATGAGAGACAATAACCCTG.

The reverse transcriptase reaction uses standard conditions utilizing SuperScript™ II H-reverse transcriptase (Invitrogen), followed by amplification of the resulting cDNA with Taq polymerase, again, using standard conditions. The resulting DNA molecules are then ligated into a self-replicating prokaryotic plasmid and introduced into E. coli cells by transformation (using standard transformation protocols).

Transformed cells are then taken through rounds of enrichment (transformed cells are allowed to grow in rich media for 1 hour at 37° C. prior to being transferred to fresh rich media supplemented with 100 μg/ml ampicillin for 6 hours at 37° C.) and selection (cells are extracted from the ampicillin media and placed into fresh rich media containing either 5 or 20 μg/ml cefotaxime and allowed to grow for 18 hours at 37° C. before being plated onto solid rich media containing either 5 or 20 μg/ml cefotaxime). Clones resistant to increasing levels of cefotaxime (eg. 5 μg/ml cefotaxime (a 250-fold increase in resistance) or 20 μg/ml cefotaxime (a 1000-fold increase in resistance) or higher are then selected.

Clones selected after the first round may then be characterized by sequencing.

### Example 12

### Generation of Mutant Nucleotide Sequences with Increased Stability and Expression for DNA and RNA Vaccines

Both RNA and DNA sequences can be used in vitro or in vivo as vaccines with dendritic cells or other cell types to elicit local or systemic immunity. However, the success of the challenge depends on the stability of the nucleotide sequence particularly with RNA approaches. The major disadvantage of using RNA for transfection is that RNA is a more labile molecule than DNA. The half-life of RNA is estimated to be approximately 5 hours in serum-free tissue culture medium but is estimated to be only a few minutes when 10% serum is present. Consequently, there are major advantages to be achieved in transfection efficiency by evolving significantly more stable, degradation resistance variations of RNA coding for the same amino acid sequence. DNA vaccine sequences can also significantly benefit by using a similar approach to increase translation efficiency and expression levels in situ post-transfection.

The following procedure would be employed to test nucleotide variants that have been selected for increased stability or expression.

Isolation and Purification of Dendritic Cells

Isolation of dendritic cells involves the separation of monocytes using a discontinuous Percoll gradient. The monocyte enriched low density fraction can be depleted of B, T, and/or, NK cells using cell specific magnetic beads (Dynal). To generate immature dendritic cells, purified monocytes can be cultured in either RPMI 1640 supplemented with glutamine (2 mM), HEPES (15 mM), and 1% NHS (Sigma) or in AIM V serum-free medium (Life Technologies), supplemented with GM-CSF (50 ng/ml) and IL-4 (100 ng/ml). TNF-a (1 ng/ml) and PGEJ (500 nM) can be used for DC maturation (Weissman et al, 2000).

In Vitro Transcription

An expression vector can be used as the base plasmid for the construction of nucleotides sequences for transfection and can also be used as the template for in vitro mRNA transcription. The luciferase gene can be used as a reporter sequence. mRNA transcription can be performed on a SmaI linearized plasmid template using either T7, T3 or SP6 RNA polymerase as previously outlined in Example 10 with the addition of a m⁷ GpppG-cap at the end of the mRNA by incubating the mix with 3 mM 5′ 7meGpppG 5′ (Integrated Sciences). Self-replicating mRNA can be used to improve vaccine efficacy. Self-replicating mRNA can be generated from linearized DNA with either T7, T3 or SP6 RNA polymerase with the transcript encoding either/and/or a leader sequence such as TEV (tobacco etch virus), a non-structural polyprotein or replicase of the Semliki Forest virus or other members of the Alphavirus genus (Liljestrom and Garoff, 1991), a reporter sequence, a poly(A) tail, or other internal or 5′ and 3′ nucleotide sequences that facilitate transcription, translation, stability or delivery. For instance, incorporation of the 5′ and 3′ untranslated regions of beta-globin mRNA greatly stabilizes RNA transfected into cells and leads to over a 1,000-fold increase in reporter gene expression in transfected cells (Mitchell and Nair, 2000).

RNA transcripts can be purified by DNase I digestion followed by purification using RNeasy RNA purification kit (Qiagen). DNA can be purified using MinElute columns (Qiagen). mRNA or plasmid DNA to be delivered into cells by complexing to Lipofectin (Life Technologies) in the presence of phosphate buffer (Kariko et al, 1998) or aliquots of the mRNA or DNA can be added directly to serum-free, washed dendritic cells, B cells, monocytes, T cells, or CD4+ T cells or other T cell for 60 min and then the cells can be resuspended in fresh medium or PBS for introduction into the appropriate host. As vaccines are believed to work by direct transfection of dendritic cells, such as the Langerhans cells within the skin or other organs, aliquots of the mRNA, mRNA/lipid complexes or DNA can also be introduced into whole organisms directly via intradermal injection, injection into the spleen or other internal organ, or direct exposure to the mucosa.

Cell Cultures

RNA or DNA sequences can be either delivered as naked nucleotide sequences, as a nucleotide/liposome (or other carrier) complex, or with a gene gun or biolistic to achieve transfection into dendritic or other cells in tissue culture. Matured cells can then be purified by either negative selection using cell separation columns or by positive selection using cell type specific magnetic beads (Dynal).

Reporter Gene Product Analyses

Luciferase enzymatic activity can be measured by lysing cells in cell culture lysis reagent (Promega, Madison, Wis.), adding luciferase substrate (Promega), and measuring light intensity with a luminometer.

Immunizations and Antigen Challenge

BALB/c mice (6-8 weeks old) or other test animals can be used to test for each vaccine or immunization route. Generally, animals can be immunized by various routes 3 times at 2-week intervals, rested for 2-3 weeks, and then challenged intravaginally or intrarectally. Intranasal immunizations with particles suspended in PBS can be performed without anesthesia, while immunizations administered intravaginally or intrarectally require anesthetized animals. Animals are kept in dorsal recumbency for 20 min. Intramuscular immunizations can be made into thigh muscle.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed, particularly in Australia, before the priority date of each claim of this application.

The invention is further described by the following numbered paragraphs:

1. A method of introducing one or more mutations during replication or transcription of a target nucleic acid molecule, the method comprising
(i) incubating the target nucleic acid molecule with a polymerase in the presence of ribavirin, or a derivative/analogue thereof, under conditions that allow the introduction of a mutation(s) during transcription or replication of the target nucleic acid, and
(ii) selecting a mutant target nucleic acid molecule or selecting for an effect of the introduced mutation(s).

2. The method of paragraph 1, wherein step (i) and/or step (ii) is performed in an in vitro or cell free system.

3. The method of paragraph 1, wherein step (i) and/or step (ii) is performed within a cell.

4. The method of any one of paragraphs 1 to 3, wherein step (ii) involves selecting a mutant target nucleic acid molecule.

5. The method of any one of paragraphs 1 to 4, wherein the mutant nucleic acid molecule has an altered property or activity.

6. The method of paragraph 5, wherein the altered property or activity is enhanced expression of an encoded polypeptide when compared to the level of expression of the polypeptide before the introduction of a mutation(s) in step (i).

7. The method of paragraph 5, wherein the altered property or activity is enhanced stability when compared to the level of stability before the introduction of a mutation(s) in step (i).

8. The method of paragraph 5, wherein the altered property or activity is altered catalytic activity when compared to the level of catalytic activity before the introduction of a mutation(s) in step (i).

9. The method of paragraph 5, wherein the altered property or activity is enhanced RNA interference activity when compared to the level of RNA interference activity before the introduction of a mutation(s) in step (i).

10. The method of paragraph 5, wherein the altered property or activity is enhanced antisense activity when compared to the level of antisense activity before the introduction of a mutation(s) in step (i).

11. The method of any one of paragraphs 1 to 4, wherein the mutant nucleic acid molecule encodes a protein with an altered property or activity.

12. A method of identifying a mutant protein with a desired property, the method comprising
(i) incubating a target nucleic acid molecule with a polymerase in the presence of ribavirin, or a derivative/analogue thereof, under conditions which allow transcription or replication of the target nucleic acid,
(ii) producing a protein encoded by a nucleic acid produced from step (i), and
(iii) screening the protein for a desired property.

13. The method of paragraph 12, wherein the nucleic acid produced from step (i) is copied in the absence of ribavirin or a derivative/analogue thereof before production of the encoded protein.

14. The method of paragraph 12 or paragraph 13, wherein the protein produced in step (ii) is associated with its encoding nucleic acid molecule.

15. The method of paragraph 14, wherein the protein is associated with its encoding RNA molecule via a ribosome complex.

16. The method of paragraph 14, wherein the protein is associated with its encoding RNA molecule via an RNA binding molecule.

17. The method of paragraph 14, wherein the protein is associated with its encoding nucleic acid molecule by virtue of association with or location within the same cell or viral particle.

18. The method of any one of paragraphs 14 to 17 which further comprises the step of recovering the encoding nucleic acid molecule.

19. The method of paragraph 18, wherein the encoding nucleic acid molecule is recovered by reverse transcription, RT-PCR amplification or PCR amplification.

20. The method of any one of paragraphs 12 to 19, wherein the protein is produced in a translation system comprising oxidised and/or reduced glutathione at a total concentration of between about 0.1 mM to about 10 mM.

21. The method of any one of paragraph 12 to 20, wherein the nucleic acid produced from step (i) is RNA and the method further comprises reverse transcribing the RNA into DNA before the protein is produced.

22. The method of paragraph 21, wherein the DNA is cloned into a suitable vector and transformed/transfected into a host cell before the protein is produced.

23. The method of any one of paragraphs 1 to 22, wherein the polymerase is a DNA dependent RNA polymerase and the target nucleic acid molecule is a DNA molecule.

24. The method of paragraph 23, wherein the DNA dependent RNA polymerase is selected from the group consisting of T7 RNA polymerase, SP6 RNA polymerase and T3 RNA polymerase.

25. The method of any one of paragraphs 1 to 20, wherein the polymerase is a DNA dependent DNA polymerase and the target nucleic acid molecule is a DNA molecule.

26. The method of paragraph 25, wherein the DNA dependent DNA polymerase is selected from the group consisting of Tth DNA polymerase, Vent DNA polymerase, Pwo polymerase, E. Coli DNA polymerase I Klenow fragment and T4 DNA polymerase.

27. The method of any one of paragraphs 1 to 20, wherein the polymerase is a RNA dependent DNA polymerase and the target nucleic acid molecule is a RNA molecule.

28. The method of paragraph 27, wherein the RNA dependent DNA polymerase is selected from the group consisting of AMV reverse transcriptase, M-MLV reverse transcriptase, SuperScript III and Tth polymerase.

29. The method of any one of paragraphs 1 to 22, wherein the polymerase is an RNA dependent RNA polymerase and the target nucleic acid molecule is a RNA molecule.

30. The method of paragraph 29, wherein the RNA dependent RNA polymerase is selected from the group consisting of Qβ replicase, Hepatitis C RdRp, Vesicular Stomatitis Virus RdRp, Turnip yellow mosaic virus replicase and RNA bacteriophage phi 6 RNA-dependent RNA polymerase.

31. The method of any one of paragraphs 1 to 30, wherein the replication or transcription is performed in combination with at least one additional mutagenic procedure.

32. The method of paragraph 31, wherein the at least one additional mutagenic procedure involves addition of a chemical mutagen.

33. A kit comprising ribavirin, or a derivative/analogue thereof, and at least one reagent required for the replication or transcription of a nucleic acid molecule.

34. The kit of paragraph 33, wherein the at least one reagent is selected from the group consisting of a polymerase, a nucleic acid molecule encoding a polymerase, a reaction buffer, and nucleosides or nucleotides.

35. The kit of paragraph 34, wherein the polymerase has reduced or deficient proof reading activity.

36. The kit of any one of paragraphs 33 to 35, wherein the kit further comprises at least one additional mutagen.

37. A kit comprising ribavirin, or a derivative/analogue thereof, and at least one additional mutagen.

38. A kit of any one of paragraphs 33 to 37 for use in a method of any one of paragraphs 1 to 32.

39. A method for identifying a mutant RNA molecule which exhibits an altered property or activity, the method comprising
(i) incubating a target RNA molecule with an RNA dependent RNA polymerase under conditions wherein the RNA dependent RNA polymerase replicates the RNA molecule but introduces a mutation(s) thereby generating a population of mutant RNA molecules; and

(ii) selecting a mutant RNA molecule that exhibits an altered property or activity.

40. The method of paragraph 39, wherein the altered property or activity is enhanced expression of an encoded polypeptide when compared to the level of expression of the polypeptide before the introduction of a mutation(s) in step (i).

41. The method of paragraph 39, wherein the altered property or activity is enhanced stability when compared to the level of stability before the introduction of a mutation(s) in step (i).

42. The method of paragraph 39, wherein the altered property or activity is altered catalytic activity when compared to the level of catalytic activity before the introduction of a mutation(s) in step (i).

43. The method of paragraph 39, wherein the altered property or activity is enhanced RNA interference activity when compared to the level of RNA interference activity before the introduction of a mutation(s) in step (i).

44. The method of paragraph 39, wherein the altered property or activity is enhanced antisense activity when compared to the level of antisense activity before the introduction of a mutation(s) in step (i).

45. The method of paragraph 39, wherein the mutation(s) introduced into the RNA molecule in step (i) does not alter the amino acid sequence of a protein encoded by the RNA molecule.

46. The method of any one of paragraphs 39 to 45, further comprising the step of transcribing a DNA construct to produce replicable RNA.

47. The method of any one of paragraphs 39 to 46, wherein step (i) and/or step (ii) is performed in an in vitro system.

48. The method of any one of paragraphs 39 to 46, wherein step (i) and/or step (ii) is performed within a cell.

49. The method of any one of paragraphs 39 to 48, wherein step (i) is performed in combination with at least one additional mutagenic procedure.

50. The method of paragraph 49, wherein the at the least one additional mutagenic procedure involves addition of a chemical mutagen.

51. The method of paragraph 50, wherein the chemical mutagen is ribavirin or a derivative/analogue thereof.

### REFERENCES

Bilenoglu et al. (2002). Brit J Haematol 119:1106-1114.
Boder and Wittrup (1997). Nature Biotech 15:553-557.
Brown and Gold (1995). Biochemistry 34:14775-14782.
Brown and Gold (1996). Proc Natl Acad Sci (USA) 93:11558-11562.
Buchholz et al. (1998). Nature Biotech 16:951-954.
Caldwell and Joyce (1992). PCR Meth Appl 2:28-33.
Cao and Parker (2001). RNA 7:1192-1212.
Chen and Georgiou (2002). Biotech Bioeng 79:496-503.
Cho et al. (1998). J Immunol Methods 220:179-188.
Cisneros et al. (1996). Gene 181:129-133.
Deiman et al. (1997). J Virol 71:5990.
Deras and Woodson (2000). J Mol Biol 296:133-144.
Dougherty and Parks (1995). Curr Opin Cell Biol 7:399-405.
Durr and Lindh (1975). Ann N Y Acad Sci 255:366-371.
Emory and Belasco (1990). J Bacteriol 172:4472-4481.
Falmm et al. (2001). RNA 7:254-265.
Felici et al. (1995). Biotech Annu Rev 1: 149-183.
Fields and Song (1989). Nature 340:245-246.
Fire et al. (1998). Nature 391:806-811.
Ghee et al. (1996). J Mol Biol 261:222-230.
Guo and Cech (2002). Nat Struct Biol 9:855-861.
Hambraeus et al. (2002). Microbiology 148:1795-1803.
Haseloff and Gerlach (1988). Nature 334:585-591.
He and Taussig (1997). Nucleic Acids Research 25:5132-5134.
Heid et al. (1996). Genome Res 6:986-994.
Helgstrand et al. (2002). Nucleic Acids Research 30:2678.
Hoogenboom (2002). Methods Mol Biol 178:1-37.
Hruska et al. (1982). Antimicrob Agents Chemother 21:125-130.
Irving et al. (1996). Immunotechnology 2:127-143.
Ito and Jacobs-Lorena (2001). J Biol Chem 276:23525-23530.
Izant and Weintraub (1984). Cell 36:1007-1015.
Izant and Weintraub (1985). Science 229(4711):345-352.
Jiang et al. (1997). Infect Immun 65:4770-4777.
Joelson et al. (1997). J Gen Virol 78:1213-1217.
Jung et al. (1998). Nature Biotechnology 16:576-580.
Kariko et al (1998). Biochim Biophys Acta 1369:320-334.
Kosinski et al. (2003). J Immunol 170:979-988.
Kramer et al. (1978). Proc Natl Acad Sci (USA) 75:5334-5338.
Leroy et al. (2002). Mol Microbiol 45:1231-1243.
Leung et al. (1989). Technique 1:11-15.
Licitra and Liu (1996). Proc Natl Acad Sci USA 93:12817-12821.
Liljestrom and Garoff (1991). Biotechnology 9:1356-1361.
Lizardi et al. (1988). Bio/Technology 6:1197-1202.
Lowman et al. (1991). Biochem 30:10832-10838.
Makeyev and Bamford (2000). The EMBO J 19:124-1133.
Marcus-Sakura (1988). Anal Biochem 172:289.
Markland et al. (2000). Antimicrob Agents Chemother 44:859-866.
Mattheakis et al. (1994). Proc. Natl. Acad. Sci. USA 91:9022-9026.
McCormick et al. (1986). N Engl J Med 314:20-26.
McHutchison et al. (1998). N Engl J Med 339:1485-1492.
Melton et al. (1984a). Nucleic Acid Res 12:7035-7056.
Melton et al. (1984b). Nucleic Acid Res 12:7057-7070.
Mitchell and Nair (2000). J Clin Invest 106:1065-1069.
Munishkin et al. (1991). J Mol Biol 221:463-472.
Nemoto et al. (1997). FEBS Lett 414:405.
Nishihara et al. (1983). J Biochem (Tokyo 93:669-674.
Ojala and Bamford (1995). Virology 207:400.
Perriman et al. (1992). Gene 113:157-163.
Rasmussen et al. (2002). J Mol Biol 323:613-628.
Roberts (1999). Current Opinion in Chemical Biology 3:268.
Roberts and Szostak (1997). PNAS 94:12297.
Rohde et al. (1995). J Mol Biol 249:754-762.
Ryabova et al. (1997). Nature Biotech 15:79-84.
Schaaf and Cidlowski (2002). Steroids 67:627-636.
Schmittgen et al. (2000). Anal Biochem 285:194-204.
Sharp (1999). Genes & Development 13:139-141.
Sharp and Li (1986). Nucleic Acids Res 14:7737-7749.
Shippy et al. (1999). Mol Biotech 12:117-129.
Sidwell et al. (1972). Science 177:705-706.
Spirin et al. (1988). Science 242:1162-1164.
Stahl and Uhlen (1997). TIBTECH 15:185-192.
Stemmer (1994). Nature 370:389-391.
Stoecklin et al. (2001). RNA 7:1578-1588.
Stolz et al. (1998). FEBS Letters 440:213-217.
Sumper & Luce (1975). Proc Natl Acad Sci USA 72:162-166.
Sutiphong et al. (1987). Mol Biol Med 4:307-322.
Vidal et al. (1996). Proc Natl Acad Sci (USA) 93:10315-10320.
Volkov and Arnold (2000). Methods Enzymol 328:447-456.
von Herrath et al. (2000). Virology 268:411-419.
Waterhouse et al. (1998). Proc Natl Acad Sci (USA) 95:13959-13964.
Weissman et al. (2000). J Immunol: 165:4710-4717.
Widersten et al. (1996). Protein Expr Purif 7:367-372.
Wittrup (2001). Curr Opin Biotech 12:395-399.
Wittwer et al. (1997). Biotechniques 22:130-131 and 134-138.
Wyde (1998). Antiviral Res 39:63-79.
Zubay (1973). Ann Rev Genetics 7:267-287.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1-29. (canceled)

30. A method of introducing one or more mutations during replication or transcription of a target nucleic acid molecule, the method comprising
(i) incubating the target nucleic acid molecule with a polymerase in the presence of ribavirin, or a derivative/analogue thereof, under conditions that allow the introduction of a mutation(s) during transcription or replication of the target nucleic acid, and
(ii) selecting a mutant target nucleic acid molecule or selecting for an effect of the introduced mutation(s).

31. The method of claim 30, wherein step (i) and/or step (ii) is performed in an in vitro or cell free system, or is performed within a cell.

32. The method of claim 30, wherein step (ii) involves selecting a mutant target nucleic acid molecule.

33. The method of claim 30, wherein the mutant nucleic acid molecule has an altered property or activity.

34. The method of claim 30, wherein the mutant nucleic acid molecule encodes a protein with an altered property or activity.

35. A method of identifying a mutant protein with a desired property, the method comprising
(i) incubating a target nucleic acid molecule with a polymerase in the presence of ribavirin, or a derivative/analogue thereof, under conditions which allow transcription or replication of the target nucleic acid,
(ii) producing a protein encoded by a nucleic acid produced from step (i), and
(iii) screening the protein for a desired property.

36. The method of claim 35, wherein the nucleic acid produced from step (i) is copied in the absence of ribavirin or a derivative/analogue thereof before production of the encoded protein.

37. The method of claim 35, wherein the protein produced in step (ii) is associated with its encoding nucleic acid molecule.

38. The method of claim 30, which further comprises the step of recovering the encoding nucleic acid molecule.

39. The method of claim 30, wherein the nucleic acid produced from step (i) is RNA and the method further comprises reverse transcribing the RNA into DNA before the protein is produced.

40. The method of claim 30, wherein the polymerase is a DNA dependent RNA polymerase and the target nucleic acid molecule is a DNA molecule.

41. The method of claim 30, wherein the polymerase is a DNA dependent DNA polymerase and the target nucleic acid molecule is a DNA molecule.

42. The method of claim 30, wherein the polymerase is a RNA dependent DNA polymerase and the target nucleic acid molecule is a RNA molecule.

43. The method of claim 30, wherein the polymerase is an RNA dependent RNA polymerase and the target nucleic acid molecule is a RNA molecule.

44. The method of claim 30, wherein the replication or transcription is performed in combination with at least one additional mutagenic procedure.

45. A kit comprising ribavirin, or a derivative/analogue thereof, and at least one reagent required for the replication or transcription of a nucleic acid molecule, and/or at least one additional mutagen.

46. The kit of claim 45, wherein the at least one reagent is selected from the group consisting of a polymerase, a nucleic acid molecule encoding a polymerase, a reaction buffer, and nucleosides or nucleotides.

47. A method for identifying a mutant RNA molecule which exhibits an altered property or activity, the method comprising
(i) incubating a target RNA molecule with an RNA dependent RNA polymerase under conditions wherein the RNA dependent RNA polymerase replicates the RNA molecule but introduces a mutation(s) thereby generating a population of mutant RNA molecules; and
(ii) selecting a mutant RNA molecule that exhibits an altered property or activity.

48. The method of claim 47, wherein the altered property or activity is enhanced expression of an encoded polypeptide when compared to the level of expression of the polypeptide before the introduction of a mutation(s) in step (i).

49. The method of claim 47, wherein the altered property or activity is enhanced stability when compared to the level of stability before the introduction of a mutation(s) in step (i).


SEQUENCE LISTING (12 sequences)

SEQ ID NO: 1 - protein, 8 aa, Artificial Sequence
  Description of Artificial Sequence Synthetic FLAG epitope
DYKDDDDK

SEQ ID NO: 2 - DNA, 48 bp, Artificial Sequence
  Description of Artificial Sequence Synthetic primer
ttactcgcggcccagccggccatggccatgtctaagacagcatcttcg

SEQ ID NO: 3 - DNA, 39 bp, Artificial Sequence
  Description of Artificial Sequence Synthetic primer
tttataatctgcggccgccgcctcgtgtagagacgcaac

SEQ ID NO: 4 - DNA, 34 bp, Artificial Sequence
  Description of Artificial Sequence Synthetic primer
cgaggcggccgcggtcatgagattatcaaaaagg

SEQ ID NO: 5 - DNA, 33 bp, Artificial Sequence
  Description of Artificial Sequence Synthetic primer
tcgagccatggctcatgagagacaataaccctg

SEQ ID NO: 6 - RNA, 64 nt, Artificial Sequence
  Description of Artificial Sequence Synthetic oligonucleotide
auggccgcauggguagaccaaacgagaaaggagcuggcaccgcauuaaccgugaaagcggccgc

SEQ ID NO: 7 - RNA, 10 nt, Artificial Sequence
  Description of Artificial Sequence Synthetic oligonucleotide
aaagccgcgg

SEQ ID NO: 8 - RNA, 62 nt, Artificial Sequence
  Description of Artificial Sequence Synthetic oligonucleotide
auggccgcauggguagaccaaacaccaagaacaacaacaaaagagacgggcgaaucacugac

SEQ ID NO: 9 - RNA, 19 nt, Artificial Sequence
  Description of Artificial Sequence Synthetic oligonucleotide
uuagugaucugagaguuga

SEQ ID NO: 10 - RNA, 42 nt, Artificial Sequence
  Description of Artificial Sequence Synthetic oligonucleotide
gcauucuauucucuuccguugggcgauuauaacuauucucug

SEQ ID NO: 11 - RNA, 49 nt, Artificial Sequence
  Description of Artificial Sequence Synthetic oligonucleotide
gugagaaaggagcuggcaccgcauuaaccgugaaagcggccgcagauua

SEQ ID NO: 12 - RNA, 16 nt, Artificial Sequence
  Description of Artificial Sequence Synthetic oligonucleotide
gaugauaaagccgcgg